(19) United States
(12) Patent Application Publication
(10) Pub. No.: US 2015/0283048 A1
(43) Pub. Date: Oct. 8, 2015

(54) TEXTILE-SPARING ANTIPERSPIRANT SPRAYS WITH HYDROXYCARBOXYLIC ACIDS

(71) Applicant: Henkel AG & Co. KGaA, Duesseldorf (DE)

(72) Inventors: Bernhard Banowski, Duesseldorf (DE); Marcus Claas, Hilden (DE); Imme Breuer, Duesseldorf (DE); Christian Kropf, Hilden (DE)

(21) Appl. No.: 14/742,858

(22) Filed: Jun. 18, 2015

Related U.S. Application Data
(63) Continuation of application No. PCT/EP2013/076657, filed on Dec. 16, 2013.

(30) Foreign Application Priority Data
Dec. 21, 2012 (DE) 10 2012 224 157.4

(51) Int. Cl. A61K 8/26; A61K 8/04; A61Q 15/00; A61K 8/35

(52) U.S. Cl. CPC A61K 8/26; A61K 8/35; A61K 8/046; A61Q 15/00; A61K 2800/30

(57) ABSTRACT

The present patent application relates to cosmetic antiperspirants for aerosol application, including at least one hydroxycarboxylic acid of the formula (HCS-1) HOOC—(CH—OH)n-CHOH—R (HCS-1), where n is an integer in the range of 1 to 10, and the substituent R is selected from —H and —COOH, wherein the hydroxycarboxylic acid may be present in free form, in salt form or as a lactone, and wherein the total amount of hydroxycarboxylic acid(s) of the formula (HCS-1), optionally in salt form and/or lactone form, is 0.3-5 wt %, preferably 0.5-3 wt %, particularly preferably 1-2.5 wt %. The antiperspirants according to the invention soil textiles to a lesser degree than known antiperspirants.

### FIELD OF THE INVENTION

The present invention generally relates to cosmetic antiperspirant sprays having a lower textile soiling effect than known antiperspirants.

### BACKGROUND OF THE INVENTION

Washing, cleaning and body care constitute a basic human need and modern industry has made ongoing efforts to meet these human needs in a variety of ways. Persistent elimination or at least reduction of body odor is especially important for daily hygiene. Numerous special deodorant or antiperspirant body care agents are known in the prior art. These agents were developed for use in areas of the body that have a high density of sweat glands, in particular in the axillary area. These products are fabricated in a wide variety of administration forms, for example, as a powder, in stick form, as an aerosol spray, pump spray, liquid and gelatinous roll-on application, cream, gel and as an impregnated flexible substrate (deodorant pads).

Cosmetic antiperspirants at any rate include at least one antiperspirant salt. In most cases, they also include at least one oil or one fatty substance and at any rate they always include a fragrance component, i.e., a perfume.

With regular application, antiperspirants can lead to definitely visible colored textile soiling. These are often yellow spots which cannot be removed even by intense washing. The formation of the spots is based on a complex interaction of ingredients of the formulation, perspiration and the detergent used. Presumably insoluble aluminum compounds are formed first and within the fiber. The yellow coloration usually occurs with a time lag and is caused at least in part by oxidation of unsaturated fatty acids which are in the form of insoluble aluminum salts. Unfortunately, different factors can interact here in an unexpected way and pronounced yellow spots may form on textiles, depending on the choice of perfume oil and detergent and the individual amount and composition of the perspiration.

Insoluble compounds that may be absorbed by a textile are formed by the interaction of detergents and antiperspirant active ingredients. These insoluble compounds form hard white residues, which are usually visible on the textile only after several cycles of soiling and washing. These white residues are insoluble in water and cannot be removed by standard washing methods. They are especially readily visible on textiles of light or dark colors. A skillful choice of additives leads to a definitely lower formation of these insoluble deposits or to a delay in their formation.

Cosmetic oils or polyols are used to mask white residues on dark textiles due to transfer of these products from skin to textile when getting dressed, for example. These masking agents can also be absorbed by a textile. Depending on the chemical composition, these masking agents can be removed only partially or not at all by a standard washing process. The hydrophobic masking agent accumulates on the textile and leads to a dark greasy/oily spot, which can even alter the haptics of the textiles in the soiled area under some circumstances. A skilled choice of additives leads to a definitely lower formation of these oily/greasy dark spots of soiling and/or a delay in their formation.

There is therefore a demand for antiperspirant formulations that can reliably prevent the formation of yellow, white and/or greasy spots.

To protect textiles from such permanent soiling, various ingredients are added in the state of the art. One additive that is commonly used is surfactants such as those disclosed in WO 2010/097205 A2. The choice of the oil components can reduce or even increase the textile soiling; see, for example, U.S. Pat. No. 5,925,338, U.S. Pat. No. 4,511,554 or U.S. Pat. No. 3,974,270.

### BRIEF SUMMARY OF THE INVENTION

Cosmetic product consisting of a package, selected from a pump spray container, a squeeze container, a spray can including at least one propellant and an antiperspirant cosmetic agent included therein for spray application, including in a cosmetically tolerable vehicle: at least one antiperspirant zirconium-free aluminum salt in a total amount of 2-40% by weight, preferably 8-35% by weight, especially preferably 10-28% by weight and extremely, preferably 12-20% by weight, wherein the amounts in percent by weight are based on the total weight of the active substance (USP), which is free of ligands and free of water of crystallization in the agent; and in addition to that at least one hydroxycarboxylic acid of the formula (HCS-1):

HOOC—(CHOH)ₙ—CHOH—R (HCS-1)

wherein n denotes an integer in the range of 1 to 10, and the substituent R is selected from —H and —COOH, wherein the hydroxycarboxylic acid may be present in free form, in salt form or as a lactone and wherein the total amount of hydroxycarboxylic acid(s) of the formula (HCS-1), optionally in salt form and/or lactone form, is 0.3-5% by weight, preferably 0.5-3% by weight, especially preferably 1-2.5% by weight, wherein the amounts given in percent by weight are each based on the total weight of the agent, without taking into account the propellant that is optionally present.

Use of at least one hydroxycarboxylic acid of the formula (HCS-1):

HOOC—(CHOH)ₙ—CHOH—R (HCS-1)

where n denotes an integer in the range of 1 to 10, and the substituent R is selected from —H and —COOH, wherein the hydroxycarboxylic acid may be present in free form, in salt form or as a lactone and wherein the hydroxycarboxylic acid is preferably present in a total amount of 0.3-5% by weight, preferably 0.5-3% by weight, especially preferably 1-2.5% by weight, in an antiperspirant cosmetic agent, including in a cosmetically tolerable vehicle, at least one antiperspirant zirconium-free aluminum salt in a total amount of 2-40% by weight, preferably 8-35% by weight, especially preferably 10-28% by weight and extremely preferably 12-20% by weight, wherein the amounts in percent by weight are based on the total weight of the active substance (USP), which is free of ligands and free of water of crystallization in the propellant-free agent, which is present there, to reduce or prevent textile discolorations and/or textile spots, wherein the amounts in percent by weight are each based on the total weight of the agent, without taking into account a propellant that might be present.

Use of at least one hydroxycarboxylic acid of the formula (HCS-1):

HOOC—(CHOH)ₙ—CHOH—R (HCS-1)

where n denotes an integer in the range of 1 to 10, and the substituent R is selected from —H and —COOH, wherein the hydroxycarboxylic acid may be present in free form, in salt form or as a lactone and wherein the hydroxycarboxylic acid is preferably present in a total amount of 0.3-5% by weight, preferably 0.5-3% by weight, especially preferably 1-2.5% by weight, in an antiperspirant cosmetic agent, including in a cosmetically tolerable vehicle, at least one zirconium-free antiperspirant aluminum salt in a total amount of 2-40% by weight, preferably 8-35% by weight, especially preferably 10-28% by weight and extremely preferably 12-20% by weight, wherein the amounts in percent by weight are based on the total weight of the active substance (USP), which is free of ligands and free of water of crystallization, in the propellant-free agent, which is present there, to reduce or prevent textile discolorations and/or textile spots, wherein the amounts in percent by weight are each based on the total weight of the agent, without taking into account a propellant that might be present.

Method for preventing or reducing textile discolorations and/or textile spots, wherein the method comprises the following method steps: a) producing an antiperspirant cosmetic agent by mixing at least one antiperspirant zirconium-free aluminum salt in a total amount of 2-40% by weight, preferably 8-35% by weight, especially preferably 10-28% by weight and extremely, preferably 12-20% by weight, wherein the amounts in percent by weight are based on the total weight of the active substance (USP), which is free of ligands and free of water of crystallization, in the agent and is present in the propellant-free agent, with a cosmetically tolerable vehicle and with at least one hydroxycarboxylic acid of the formula (HCS-1):

HOOC—(CHOH)ₙ—CHOH—R (HCS-1)

where n denotes an integer in the range of 1 to 10, and the substituent R is selected from —H and —COOH, wherein the hydroxycarboxylic acid may be present in free form, in salt form or as a lactone and wherein the hydroxycarboxylic acid is preferably present in a total amount of 0.3-5% by weight, preferably 0.5-3% by weight, especially preferably 1-2.5% by weight, wherein the amounts by weight are each based on the total weight of the agent; b) bottling the agent in a package, selected from a pump spray container, a squeeze container and a spray can including at least one propellant; c) applying the antiperspirant cosmetic agent to the skin in particular to the axillary skin; d) wearing an item of textile clothing over the treated skin; and e) washing the item of textile clothing, in particular washing repeatedly the item of textile clothing wherein after washing, in particular after washing several times, reduced textile discolorations and/or textile spots or none at all appear.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

An object of the present invention was to provide cosmetic antiperspirant sprays that contain a perspiration-inhibiting zirconium-free aluminum salt and result in permanent textile discoloration only to a greatly reduced extent or not at all.

It has now surprisingly been found that the addition of at least one hydroxycarboxylic acid of the formula (HCS-1), HOOC—(CHOH)ₙ—CHOH—R, where n denotes an integer in the range of 1 to 10, and the substituent R is selected from —H and —COOH, wherein the hydroxycarboxylic acid may be present in free form, in salt from —H or as a lactone and wherein the total amount of hydroxycarboxylic acid(s) of the formula (HCS-1), optionally in salt form and/or lactone form, is 0.3-5% by weight, preferably 0.5-3% by weight, especially preferably 1-2.5% by weight. They solve the objects of the invention as formulated very well and are excellently suited as discoloration inhibitors for use in cosmetic antiperspirants.

Furthermore, other desirable features and characteristics of the present invention will become apparent from the subsequent detailed description of the invention and the appended claims, taken in conjunction with the accompanying drawings and this background of the invention.

A first subject matter of the present application is a cosmetic product consisting of:
i. a package, selected from a pump spray container, a squeeze container and a spray can, including at least one propellant as well as
ii. an antiperspirant cosmetic agent included therein for spray application, including, in a cosmetically tolerable vehicle:
   a) at least one zirconium-free aluminum salt antiperspirant in a total amount of 2-40% by weight, preferably 8-35% by weight, especially 10-28% by weight and extremely preferably 12-20% by weight, wherein the amounts in percent by weight are based on the total weight of the active substance (USP), which is free of ligands and free of water of crystallization, in the agent, and in addition to that,
   b) at least one hydroxycarboxylic acid of the formula (HCS-1)

HOOC—(CHOH)ₙ—CHOH—R (HCS-1)
where n denotes an integer in the range of 1 to 10, and the substituent R is selected from —H and —COOH, wherein the hydroxycarboxylic acid may be present in free form, in salt form or as a lactone and wherein the total amount of hydroxycarboxylic acid(s) of the formula (HCS-1), optionally in salt form and/or lactone form, is 0.3-5% by weight, preferably 0.5-3% by weight, especially preferably 1-2.5% by weight,
   wherein the amounts given in percent by weight (wt %) are each based on the total weight of the agent, without taking into account the propellant that is optionally present.

Another subject matter of the present patent application is the use of at least one hydroxycarboxylic acid of the formula (HCS-1)

HOOC—(CHOH)ₙ—CHOH—R (HCS-1)

where n denotes an integer in the range of 1 to 10, and the substituent R is selected from —H and —COOH, wherein the hydroxycarboxylic acid may be present in free form, in salt form or as a lactone and wherein the hydroxycarboxylic acid is preferably present in a total amount of 0.3-5% by weight, preferably 0.5-3% by weight, especially preferably 1-2.5% by weight,
in an antiperspirant cosmetic agent, including, in a cosmetically tolerable vehicle, at least one zirconium-free antiperspirant aluminum salt in a total amount of 2-40% by weight, preferably 8-35% by weight, especially preferably 10-28% by weight and extremely preferably 12-20% by weight, wherein the amounts in percent by weight are based on the total weight of the active substance (USP), which is free of ligands and free of water of crystallization, in the composition, to reduce or prevent discoloration of textiles and/or spots on textiles, wherein the amounts in percent by weight are each based on the total weight of the agent.

With regard to the preferred embodiments of use according to the invention, what is said below about the agents according to the invention also applies mutatis mutandis.

It should be pointed out here that the amount, given in percent by weight, inasmuch as it is not given otherwise, is based on the total weight of the agent according to the invention, without taking into account a propellant that may optionally be present.

The terms “agent according to the invention” and “composition according to the invention” are used as synonyms in the present patent application.

“Standard conditions” are understood in the sense of the present patent application to refer to a temperature of 20° C. and a pressure of 1013 mbar. Melting point data is also based on a pressure of 1013 mbar.

Preferred agents according to the invention are characterized in that the at least one hydroxycarboxylic acid of the formula (HCS-1) is selected from the compounds of the formula (HCS-1), in which n denotes an integer selected from 1, 2, 3, 4 and 5. Hydroxycarboxylic acids of the formula (HCS-1), where n=4 and R=—COOH as well as n=4 and R=—H are particularly preferred.

Additional preferred agents according to the invention are characterized in that the at least one hydroxycarboxylic acid of the formula (HCS-1) is selected from hydroxymalonic acid (tartronic acid), dihydroxysuccinic acid, trihydroxyglutaric acid, tetrahydroxyadipic acid, gluconic acid (R=—H and n=4), glucoheptonic acid (R=—H and n=5), mannonic acid (R=—H and n=4) and ribaric acid (R=—COOH and n=3), all enantiomers of the aforementioned acids as well as the physiologically tolerable salts and the lactones of these acids, in particular D-gluconic acid 5-lactone.

Enantiomers of dihydroxysuccinic acid (R=—COOH and n=1) that are preferred according to the invention are selected from tartaric acid, erythraric acid (mesotartaric acid), L-threaric acid ((+)-tartaric acid), D(−)-tartaric acid and pyruvic acid (racemic tartaric acid). Extremely preferred enantiomers of tetrahydroadipic acid according to the invention are selected from glucaric acid (R=—COOH and n=4), gularic acid (R=—COOH and n=4) and galactaric acid (mucic acid, R=—COOH and n=4) and mixtures thereof, as well as of the salts of these acids in particular the sodium salts and the potassium salts of glucaric acid, gularic acid and/or galactaric acid.

Additional preferred agents according to the invention are characterized in that the physiologically tolerable salts of the at least one hydroxycarboxylic acid of the formula (HCS-1) are selected from the sodium, potassium, magnesium, calcium, manganese, zinc and aluminum salts of the at least one hydroxycarboxylic acid of the formula (HCS-1). The (monovalent) sodium and potassium salts of gluconic acid and of tetrahydroxyadipic acids, in particular glucaric acid, galactaric acid and gularic acid are particularly preferred. Sodium gluconate, potassium glucarate, potassium galactarate and potassium gularate as well as mixtures thereof are extremely preferred.

In addition, the sodium salts or potassium salts of the aforementioned hydroxycarboxylic acids of the formula (HCS-1) are especially preferred.

In one extremely preferred embodiment, if the free acid and/or a salt of the free acid is used, such a hydroxycarboxylic acid of the formula (HCS-1), in which R=—COOH, is preferred.

The lactones of the hydroxycarboxylic acids of the formula (HCS-1) are extremely preferred. The lactones are cyclic esters (oxygen heterocycles), which can also be interpreted as “internal esters” of hydroxycarboxylic acids. A lactone of the hydroxycarboxylic acids of the formula (HCS-1) that is particularly preferred according to the invention is D-gluconic acid 5-lactone, which is also known as glucono-δ-lactone, gluconic acid δ-lactone or gluconolactone.

If the hydroxycarboxylic acid of the formula (HCS-1) is an optically active substance, then all the enantiomers of the acid are suitable according to the invention. The naturally occurring enantiomers are particularly preferred.

Hydroxycarboxylic acids of the formula (HCS-1) that are particularly preferred according to the invention are selected from glucaric acid (R=—COOH and n=4), gularic acid (R=—COOH and n=4) and galactaric acid (mucic acid, R=—COOH and n=4) and mixtures thereof, as well as being selected from the salts of these acids in particular the sodium salts and the potassium salts of glutaric acid, gularic acid and/or galactaric acid as well as the D-gluconic acid 5-lactone and mixtures of the aforementioned compounds.

The at least one hydroxycarboxylic acid of the formula (HCS-1) is incorporated into the composition separately from the antiperspirant aluminum salt. The at least one hydroxycarboxylic acid of the formula (HCS-1) is not an ingredient of the antiperspirant aluminum salt, for example, as a complex ligand.

The agents according to the invention include a cosmetically tolerable vehicle. The cosmetically tolerable vehicle is preferably liquid under standard conditions (20° C., 1013 mbar) according to the invention. Additional cosmetically tolerable vehicles that are preferred according to the invention include at least one cosmetic oil which is not a fragrance and not an essential oil.

The cosmetic oils that are liquid under standard conditions are not miscible with water.

When a cosmetic oil is mentioned in the present patent application, it is always a cosmetic oil that is not a fragrance and is not an essential oil, is liquid under standard conditions and is not miscible with water.

It has surprisingly been found that the textile discoloration-reducing or -preventing effect of the hydroxycarboxylic acid of the formula (HCS-1) is manifested particularly well in agents including 0 to max. 10% by weight free water.

In a first preferred embodiment of the invention, the agent according to the invention includes 0 to max. 10% by weight free water, preferably 0 to max. 5% by weight free water. The water of crystallization, hydration water or molecularly bound water, contained in the ingredients used, in particular in the antiperspirant active ingredients is not free water in the sense of the present patent application and is therefore not taken into account in calculation of the amount of water.

In another embodiment of the invention, the agent according to the invention includes free water in a total amount of 15-96% by weight, preferably 25-80% by weight, especially preferably 30-70% by weight, extremely preferably 40-60% by weight, each based on the total weight of the agent according to the invention, without having to take into account a propellant that is optionally present.

### Antiperspirant Active Ingredients

Compositions according to the invention include at least one antiperspirant zirconium-free aluminum salt as the antiperspirant active ingredient in a total amount of 2-40% by weight, preferably 8-35% by weight, especially preferably 10-28% by weight, and extremely preferably 12-20% by weight, wherein the amounts in percent by weight are based on the total weight of the substance (USP), which is free of ligands and free of water of crystallization, in the composition, without taking into account a propellant that might be present.

The antiperspirant aluminum salts are preferably selected from the water-soluble astringent organic and inorganic salts of aluminum. Aluminosilicates and zeolites are not counted with the antiperspirant active ingredients according to the invention. According to the invention, water solubility is understood to be a solubility of at least 3% by weight at 20° C. In other words, amounts of at least 3 g of the antiperspirant active ingredient are soluble in 97 g water at 20° C.

Particularly preferred antiperspirant active ingredients are selected from aluminum chlorohydrate, in particular aluminum chlorohydrate with the general formula [Al₂(OH)₅Cl.1-6H₂O]ₙ, preferably [Al₂(OH)₅Cl.2-3H₂O]ₙ, which may be present in an unactivated form or in an activated (depolymerized) form, as well as aluminum chlorohydrate with the general formula [Al₂(OH)₅Cl.1-6H₂O]ₙ, preferably [Al₂(OH)₅Cl.2-3H₂O]ₙ, which may be present in an unactivated form or in an activated (depolymerized) form.

The preparation of preferred antiperspirant active ingredients is disclosed, for example, in U.S. Pat. No. 3,887,692, U.S. Pat. No. 3,904,741, U.S. Pat. No. 4,359,456, GB 2048229 and GB 1347950.

Also preferred are aluminum sesquichlorohydrate, aluminum dichlorohydrate, aluminum chlorohydrex propylene glycol (PG) or aluminum chlorohydrex polyethylene glycol (PEG), aluminum glycol complexes, e.g., aluminum or aluminum zirconium propylene glycol complexes, aluminum sesquichlorohydrex PG or aluminum sesquichlorohydrex PEG, aluminum PG dichlorohydrex or aluminum PEG-dichlorohydrex, aluminum hydroxide, potassium aluminum sulfate (KAl(SO₄)₂.12H₂O, alum), aluminum undecylenoyl collagen amino acid, sodium aluminum lactate+aluminum sulfate, sodium aluminum chlorohydroxy lactate, aluminum bromohydrate, aluminum chloride, aluminum salts of lipoamino acids, aluminum sulfate, aluminum lactate, aluminum chlorohydroxy allantoinate and sodium-aluminum chlorohydroxy lactate.

According to the invention, antiperspirant active ingredients that are particularly preferred are selected from so-called “activated” aluminum salts, which are also known as antiperspirant active ingredients “with enhanced activity”. Such active ingredients are known in the prior art and are also available commercially. Their production is disclosed in GB 2048229, U.S. Pat. No. 4,775,528 and U.S. Pat. No. 6,010,688, for example. Activated aluminum salts are usually created by heat treatment of a relatively dilute solution of the salt (e.g., approx. 10% by weight salt) to increase its HPLC peak 4-to-peak 3 area ratio. The activated salt can then be dried to form a powder, in particular being spray-dried. In addition to spray drying, drum drying is also suitable, for example.

Activated aluminum salts typically have an HPLC peak 4-to-peak 3 area ratio of at least 0.4, preferably at least 0.7, especially preferably at least 0.9, wherein at least 70% of the aluminum is to be assigned to this peak.

Activated aluminum salts need not necessary be used as a spray-dried powder. According to the invention, antiperspirant active ingredients that are also preferred are nonaqueous solutions or solubilisates of an activated antiperspirant aluminum salt, for example, according to U.S. Pat. No. 6,010,688, which are stabilized against the loss of activation to prevent rapid degradation of the HPLC peak 4-to-peak 3 area ratio of the salt, being stabilized by the addition of an effective amount of a polyhydric alcohol, which has 3 to 6 carbon atoms and 3 to 6 hydroxyl groups, preferably propylene glycol, sorbitol and pentaerythritol. For example, compositions including the following in percent by weight (USP) are preferred: 18-45% by weight of an activated aluminum salt, 55-82% by weight of at least one anhydrous polyhydric alcohol with 3 to 6 carbon atoms and 3 to 6 hydroxyl groups, preferably 1,2-propylene glycol, 1,3-butylene glycol, diethylene glycol, dipropylene glycol, glycerol, sorbitol and pentaerythritol, especially preferably 1,2-propylene glycol.

Complexes of activated antiperspirant aluminum salts with a polyhydric alcohol including 20 to 50% by weight, especially preferably 20 to 42% by weight activated antiperspirant aluminum salt and 2 to 16% by weight molecularly bound water are especially preferred, wherein up to 100% by weight of the remainder is at least one polyhydric alcohol with 3 to 6 carbon atoms and 3 to 6 hydroxyl groups. Propylene glycol, propylene glycol/sorbitol mixtures and propylene glycol/pentaerythritol mixtures are preferred alcohols of this type. Such preferred complexes of an activated antiperspirant aluminum salt with a polyhydric alcohol according to the invention are disclosed in U.S. Pat. No. 5,643,558 and U.S. Pat. No. 6,245,325, for example.

Additional preferred antiperspirant active ingredients are basic calcium aluminum salts, such as those disclosed in U.S. Pat. No. 2,571,030, for example. These salts are produced by reacting calcium carbonate with aluminum chlorhydroxide or aluminum chloride and aluminum powder or by adding calcium chloride dihydrate to aluminum chlorohydroxide.

Additional preferred antiperspirant active ingredients are activated salts such as those disclosed in U.S. Pat. No. 6,245,325 or U.S. Pat. No. 6,042,816, for example, including 5-78% by weight (USP) of an activated antiperspirant aluminum salt, an aluminum acid or a hydroxyalkanoic acid in such an amount to supply an (amino acid or hydroxyalkanoic acid)-to-Al weight ratio of 2:1-1:20 and preferably 1:1 to 1:10 as well as a water-soluble calcium salt in such an amount to supply a Ca:(Al+Zr) weight ratio of 1:1-1:28 and preferably 1:2-1:25, especially preferably solid activated antiperspirant salt compositions, for example, according to U.S. Pat. No. 6,245,325 or U.S. Pat. No. 6,042,816, including 48-78% by weight (USP), preferably 66-75% by weight of an activated aluminum salt and 1-16% by weight, preferably 4-13% by weight molecularly bound water (water of hydration), in addition, enough water-soluble calcium salt so that the Ca:Al weight ratio is 1:1-1:28, preferably 1:2-1:25, and enough amino acid, so that the amino acid-to-Al weight ratio is 2:1-1:20, preferably 1:1-1:10.

Additional particularly preferred solid antiperspirant activated salt compositions according to U.S. Pat. No. 6,245,325 or U.S. Pat. No. 6,042,816, for example, include 48-78% by weight (USP), preferably 66-75% by weight, of an activated aluminum salt and 1-16% by weight, preferably 4-13% by weight molecularly bound water (water of hydration), in addition enough water-soluble calcium salt so that the Ca:Al weight ratio is 1:1-1:28, preferably 1:2-1:25, and enough glycine, so that the glycine to Al weight ratio is 2:1-1:20, preferably 1:1-1:10.

Especially preferred solid antiperspirant activated salt compositions, for example, according to U.S. Pat. No. 6,245,325 or U.S. Pat. No. 6,042,816, include 48-78% by weight (USP), preferably 66-75% by weight of an activated aluminum salt and 1-16% by weight, preferably 4-13% by weight, molecularly bound water, in addition, enough water-soluble calcium salt, so that the Ca:Al weight ratio is 1:1-1:28, preferably 1:2-1:25, and enough hydroxyalkanoic acid, so that the hydroxyalkanoic acid-to-Al weight ratio is 2:1-1:20, preferably 1:1-1:10.

For stabilization of the antiperspirant salts, preferred amino acids are selected from glycine, alanine, leucine, isoleucine, β-alanine, valine, cysteine, serine, tryptophan, phenylalanine, methionine, β-amino-n-butanoic acid and γ-amino-n-butanoic acid and the salts thereof, each in the d form, the 1 form and the dl form. Glycine is especially preferred.

Preferred hydroxyalkanoic acids for stabilization of the antiperspirant salts are selected from glycolic acid and lactic acid.

Additional preferred activated aluminum salts are those of the general formula Al₂(OH)₆₋ₐXa, where X is Cl, Br, I or NO₃ and “a” has a value of 0.3 to 5, preferably 0.8 to 2.5 and especially preferably 1 to 2, so that the molar ratio of Al:X is 0.9:1 to 2.1:1, as disclosed in U.S. Pat. No. 6,074,632, for example. These salts generally include some water of hydration bound associatively, typically 1 to 6 mol water per mol salt. Aluminum chlorohydrate is particularly preferred (i.e., X is Cl in the aforementioned formula) and specifically 5/6 basic aluminum chlorohydrate, in which “a” is 1, so that the molar ratio of aluminum to chlorine is 1.9:1 to 2.1:1.

Especially preferred zirconium-free aluminum salts according to the invention have a molar metal-to-chloride ratio of 1.9-2.1. Especially preferred zirconium-free aluminum sesquichlorohydrates according to the invention have a molar metal-to-chloride ratio of 1.5:1-1.8:1.

The agents according to the invention are preferably in the form of a suspension of undissolved antiperspirant active ingredient in an oil. Another preferred form of administration is a sprayable water-in-oil emulsion, which is preferably sprayed by means of a propellant. Another preferred form of administration is a sprayable oil-in-water emulsion, which is preferably sprayed as a pump spray. The agents according to the invention are fabricated either as a product for use as an aerosol; in other words, they are packaged in a pressurized container from which they are sprayed with the help of a propellant. In addition, the agents according to the invention may also be sprayed as a propellant gas-free pump spray.

The composition is applied with a spray device. In a container, these spray devices contain a filling of the liquid, viscous-flowable suspension or powdered antiperspirant according to the invention. The filling may be under the pressure of a propellant (compressed gas cans, compressed gas packages, aerosol packages) or it may be a mechanically operated pump atomizer without propellant gas (pump sprays). The containers have a removal device, preferably in the form of valves, which facilitate the removal of the contents in the form of a mist, smoke, foam, powder, paste or stream of liquid. The containers for the spray devices may be cylindrical vessels made of metal (aluminum, tin plate, volume capacity preferably max. 1000 mL) in particular, protected and/or shatterproof glass or plastic (volume capacity preferably max. 220 mL) and/or shatterproof glass or plastic (volume capacity preferably 50-400 mL). Creamy, gelatinous, pasty and liquid agents can be packaged in pump, spray or squeeze dispensers, in particular also in multichamber pump, multichamber spray or multichamber squeeze dispensers. The packaging for the agents according to the invention may be opaque, transparent or translucent.

In the sense of the invention, the concept of “liquid” also includes any solid-state dispersions in liquids.

In a preferred embodiment, the agents according to the invention are present as water-in-oil emulsion which preferably includes at least one hydroxycarboxylic acid of the formula HOOC—(CHOH)ₙ—CHOH—R (HCS-1), where n is an integer in the range of 1 to 10, and the substituent R is selected from —H and —COOH, in a total amount of 0.3-5% by weight, preferably 0.5-3% by weight, especially preferably 1-2.5% by weight, wherein the hydroxycarboxylic acid may be present in free form, in salt form or as a lactone, preferably D-gluconic acid 5-lactone. In another preferred embodiment, the agents according to the invention are in the form of an oil-in-water emulsion, which includes at least one hydroxycarboxylic acid of the formula HOOC—(CHOH)ₙ—CHOH—R (HCS-1), where n is an integer in the range of 1 to 10, and the substituent R is selected from —H and —COOH, in a total amount of 0.3-5% by weight, preferably 0.5-3% by weight, especially preferably 1-2.5% by weight, wherein the hydroxycarboxylic acid may be present in free form, in salt form or as a lactone, preferably including D-gluconic acid 5-lactone.

In another preferred embodiment, the agents according to the invention are characterized in that they are an antiperspirant water-in-oil emulsion including 20-90% by weight, preferably 25-55% by weight, especially preferably 30-50% by weight, extremely preferably 35-45% by weight water, at least one emulsifier and at least one cosmetic fat or oil, wherein the amounts in percent by weight are each based on the total weight of the agent according to the invention, without taking into account a propellant that might be present.

In another preferred embodiment, the agents according to the invention are characterized in that they are an antiperspirant oil-in-water emulsion including, in a total amount of 0.3-5% by weight, preferably 0.5-3% by weight, especially preferably 1-2.5% by weight, at least one hydroxycarboxylic acid of the formula HOOC—(CHOH)ₙ—CHOH—R (HCS-1), where n is an integer in the range of 1 to 10, and the substituent R is selected from —H and —COOH, wherein the hydroxycarboxylic acid may be present in free form, in salt form or as a lactone, preferably D-gluconic acid 5-lactone, 15-90% by weight, preferably 25-55% by weight, especially preferably 30-50% by weight, extremely preferably 35-45% by weight water, at least one emulsifier and at least one cosmetic fat or oil, wherein the amounts in percent by weight are each based on the total weight of the agent according to the invention, without taking into account a propellant that might be present.

In another preferred embodiment, the agents according to the invention are characterized in that they include, in a total amount of 0.3-5% by weight, preferably 0.5-3% by weight, especially preferably 1-2.5% by weight, at least one hydroxycarboxylic acid of the formula HOOC—(CHOH)ₙ—CHOH—R (HCS-1), where n is an integer in the range of 1 to 10, and the substituent R is selected from —H and —COOH, wherein the hydroxycarboxylic acid may be present in free form, in salt form or as a lactone, preferably D-gluconic acid 5-lactone, water in a total amount of 15-90% by weight, preferably 25-70% by weight, especially preferably 30-55% by weight, extremely preferably 35-05% by weight, additionally, ethanol in a total amount of 5-50% by weight, preferably 10-40% by weight, especially preferably 15-35% by weight, extremely preferably 20-30% by weight, and at least one substance that forms a hydrogel in a total amount of 0.01-2% by weight, preferably 0.1-1% by weight, preferably 0.2-0.7% by weight, extremely preferably 0.3-0.5% by weight, wherein the amounts in percent by weight are each based on the total weight of the agent according to the invention, without taking into account a propellant that might be present.

In another preferred embodiment, the agents according to the invention are characterized in that they are present as a water-in-oil emulsion and include in a total amount of 0.3-5% by weight, preferably 0.5-3% by weight, especially preferably 1-2.5% by weight, at least one hydroxycarboxylic acid of the formula HOOC—(CHOH)ₙ—CHOH—R (HCS-1), where n is an integer in the range of 1 to 10, and the substituent R is selected from —H and —COOH, wherein the hydroxycarboxylic acid may be present in free form, in salt form or as a lactone, preferably D-gluconic acid 5-lactone, water in a total amount of 15-90% by weight, preferably 25-60% by weight, especially preferably 30-55% by weight, extremely preferably 35-50% by weight, wherein the amounts by weight are each based on the total weight of the agent according to the invention, without taking into account a propellant that might be present.

In another preferred embodiment the agents according to the invention are characterized in that they are present as an oil-in-water emulsion and include in a total amount of 0.3-5% by weight, preferably 0.5-3% by weight, especially preferably 1-2.5% by weight, at least one hydroxycarboxylic acid of the formula HOOC—(CHOH)ₙ—CHOH—R (HCS-1), where n is an integer in the range of 1 to 10, and the substituent R is selected from —H and —COOH, wherein the hydroxycarboxylic acid may be present in free form, in salt form or as a lactone, preferably D-gluconic acid 5-lactone, water in a total amount of 15-90% by weight, preferably 25-75% by weight, especially preferably 30-60% by weight, extremely preferably 35-50% by weight water, in addition ethanol in a total amount of 1-50% by weight, preferably 10-40% by weight, especially preferably 15-35% by weight, extremely preferably 20-30% by weight, wherein the amounts in percent by weight are each based on the total weight of the agent according to the invention, without taking into account a propellant that might be present.

Preferred substances that form a hydrogel according to the invention are selected from cellulose ethers, especially hydroxyalkyl celluloses, in particular hydroxypropyl cellulose, hydroxypropylmethyl cellulose, hydroxyethyl cellulose, carboxymethyl cellulose, cetylhydroxyethylcellulose, hydroxybutyl methyl cellulose, methyl hydroxyethyl cellulose, additionally, xanthan gum, sclerotium gum, succinoglycans, polygalactomannans, in particular guar gums and locust bean gum, in particular guar gum and locust bean gum itself and the nonionic hydroxyalkyl guar derivatives and locust bean gum derivatives, such as hydroxypropyl guar, carboxymethylhydroxypropyl guar, hydroxypropylmethyl guar, hydroxyethyl guar and carboxymethyl guar, additionally, pectins, agar, carrageenans, gum tragacanth, gum arabic, karaya gum, taxa gum, gellan, gelatins, casein, propylene glycol alginate, alginic acids and salts thereof, in particular sodium alginate, potassium alginate and calcium alginate, additionally, polyvinyl pyrrolidones, polyvinyl alcohols, polyacrylamides, additionally—although less preferably—physically modified starches (e.g., by pregelatinization) and/or chemically modified starches, in particular hydroxypropylated starch phosphates and octenyl starch succinates and their aluminum, calcium or sodium salts, additionally—also less preferably—acrylic acid acrylate copolymers, acrylic acid acrylamide copolymers, acrylic acid vinyl pyrrolidone copolymers, acrylic acid vinyl formamide copolymers and polyacrylates. Especially preferred hydrogel-forming agents are selected from cellulose ethers, especially from hydroxyalkyl celluloses, in particular from hydroxypropyl cellulose, hydroxypropylmethyl cellulose, hydroxyethyl cellulose, carboxymethyl cellulose, cetylhydroxyethylcellulose, hydroxybutyl methyl cellulose and methyl hydroxyethyl cellulose as well as mixtures thereof. An extremely preferred hydrogel-forming agent is hydroxyethyl cellulose.

To further support the discoloration-inhibiting effect of the at least one dicarboxy pyridine compound (DCP-1), in particular D-gluconic acid 5-lactone, it may be advantageous to add at least one chelating agent selected from ethylenediaminetetraacetic acid (EDTA) and the salts thereof as well as nitrilotriacetic acid (NTA) and mixtures of these substances to the agents according to the invention in a total amount of 0.01-0.05% by weight, preferably 0.02-0.3% by weight, especially preferably 0.05-0.1% by weight.

Additional preferred agents according to the invention are therefore characterized in that, additionally, at least one chelating agent selected from ethylenediaminetetraacetic acid (EDTA) and the salts thereof as well as from nitrilotriacetic acid (NTA) and mixtures of these substances are present in a total amount of 0.01-0.5% by weight, preferably 0.02-0.3% by weight, especially preferably 0.05-0.1% by weight, wherein the amounts in percent by weight are each based on the total weight of the agent, without taking into account a propellant that might be present.

Additional preferred compositions according to the invention optionally include at least one deodorant active ingredient in a total amount of 0.0001-40% by weight, preferably 0.2-20% by weight, especially preferably 1-15% by weight, extremely preferably 1.5-5% by weight, wherein the amounts by weight are based on the total weight of the composition, without taking into account a propellant that might be present.

According to the invention ethanol, is not considered to be a deodorant active ingredient but instead, if present at all, is present only as an ingredient of the vehicle.

In a preferred embodiment, the agents according to the invention include at least one silver salt as the deodorizing active ingredient, preferably selected from silver sulfate, silver nitrate, silver citrate, silver dihydrogen citrate, silver lactate, silver acetate, silver malate, silver succinate, silver tartrate, silver mandelate, silver salicylate, silver gluconate, silver adipate and silver galactarate as well as mixtures of these salts. Extremely preferred are silver sulfate, silver citrate, silver dihydrogen citrate and silver lactate as well as mixtures of these salts.

Additional preferred compositions according to the invention include at least one silver salt, which is preferably selected from silver sulfate, silver nitrate, silver citrate, silver dihydrogen citrate, silver lactate, silver acetate, silver malate, silver succinate, silver tartrate, silver mandelate, silver salicylate, silver gluconate, silver adipate and silver galactarate as well as mixtures of these salts in amounts such that of silver is present in a total amount of 1-100 ppm, preferably 2-50 ppm, especially preferably 5-20 ppm, extremely preferably 7-10 ppm, each based on the weight of the propellant-free composition. Based on the molecular weights of silver (107.87 g/mol) and the respective silver salts—silver lactate, for example, has a molecular weight of 196.94 g/mol—the amount of silver salt(s) required can be calculated accordingly.

In another preferred embodiment, the agents according to the invention include at least one aromatic alcohol of the structure (AA-1) as the deodorant active ingredient:

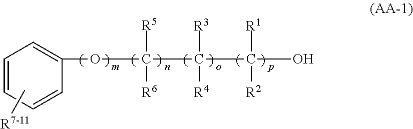

wherein
the radicals R¹ to R⁶, independently of one another, may be a hydrogen atom, an alkyl group with 1 to 10 carbon atoms, which may be linear or branched and substituted, with OH groups or alkoxy groups with 1 to 5 carbon atoms, or an alkenyl group with 2 to 10 carbon atoms, which may be linear or branched and substituted with OH groups or alkoxy groups with 1 to 5 carbon atoms,
the radicals R⁷ to R¹¹ independently of one another denote a hydrogen, a halogen, in particular a chlorine or an alkyl group with 1 to 10 carbon atoms, which may be linear or branched and substituted with OH groups or alkoxy groups with 1 to 5 carbon atoms, in particular with a methoxy group,
m=0 or 1, n, o, p=independently of one another, integers from 0 to 10 wherein at least one of the values n, o, p≠0.

Especially preferred products according to the invention include at least one alcohol AA-1 as described above, which is selected from anise alcohol, 2-methyl-5-phenylpentan-1-ol, 1,1-dimethyl-3-phenylpropan-1-ol, benzyl alcohol, 2-phenylethan-1-ol, 3-phenylpropan-1-ol, 4-phenylbutan-1-ol, 5-phenylpentan-1-ol, 2-benzylheptan-1-ol, 2,2-dimethyl-3-phenylpropan-1-ol, 2,2-dimethyl-3-(3′-methylphenyl)propan-1-ol, 2-ethyl-3-phenylpropan-1-ol, 2-ethyl-3-(3′-methylphenyl)propan-1-ol, 3-(3′-chloro-phenyl)-2-ethylpropan-1-ol, 342′-chlorphenyl)-2-ethylpropan-1-ol, 3-(4′-chlorophenyl)-2-ethylpropan-1-ol, 3-(3′,4′-dichlorphenyl)-2-ethylpropan-1-ol, 2-ethyl-3-(2′-methyl-phenyl)propan-1-ol, 2-ethyl-3-(4′-methylphenyl)propan-1-ol, 3-(3′,4′-dimethylphenyl)-2-ethylpropan-1-ol, 2-ethyl-3-(4′-methoxyphenyl)propan-1-ol, 3-(3′,4′-dimethoxy-phenyl)-2-ethylpropan-1-ol, 2-allyl-3-phenylpropan-1-ol and 2-n-pentyl-3-phenylpropan-1-ol as well as mixtures thereof. Extremely preferred is 2-benzylheptan-1-ol as well as mixtures of 2-benzylheptan-1-ol and phenoxyethanol. Additional particularly preferred compositions according to the invention include at least one alcohol AA-1 as described above in a total amount of 0.05-10% by weight, preferably 0.1-5% by weight, especially preferably 0.2-2% by weight, extremely preferably 0.3-1.5% by weight, each based on the weight of the composition, without taking into account a propellant that might be present. Extremely preferred agents according to the invention include 2-benzylheptan-1-ol in a total amount of 0.05-1.5% by weight, preferably 0.1-1% by weight, especially preferably 0.2-0.5% by weight, each based on the weight of the composition, without taking into account a propellant that might be present.

In another preferred embodiment, the agents according to the invention include as the deodorant active ingredient at least one 1,2-alkanediol with 5 to 12 carbon atoms, which is described by the formula HO—CH₂—CH(OH)—(CH₂)ₙ—CH₃ in which n stands for the numbers 2, 3, 4, 5, 6, 7, 8 or 9, as well as mixtures of these 1,2-alkanediols. Especially preferred 1,2-alkanediols with 5 to 12 carbon atoms according to the invention are selected from 1,2-pentanediol, 1,2-hexanediol, 1,2-octanediol, 1,2-decanediol and mixtures thereof. A most especially preferred combination according to the invention is mixtures of 1,2-hexanediol and 1,2-octanediol, preferably in a weight ratio of 10:1 to 1:10, more preferably of 5:1 to 1:5, especially preferably in a weight ratio of 1:1.

Preferred agents according to the invention include at least one 1,2-alkanediol with 5 to 12 carbon atoms, which is described by the formula HO—CH₂—CH(OH)—(CH₂)ₙ—CH₃ in which n stands for the numbers 2, 3, 4, 5, 6, 7, 8 or 9 in a total amount of 0.2-15% by weight, preferably 0.3-10% by weight, especially preferably 0.4-5% by weight and extremely preferably 0.5-2% by weight, each based on the weight of the composition, without taking into account a propellant that may be present. Extremely preferred agents according to the invention include 0.2-0.5% by weight 1,2-hexanediol and 0.2-0.5% by weight 1,2-octanediol, each based on the weight of the composition, without taking into account a propellant that might be present.

Additional preferred agents according to the invention are characterized in that they include the deodorant active ingredient 3-(2-ethylhexyloxy)-1,2-propanediol, preferably in a total amount of 0.05-5% by weight, preferably 0.1-2% by weight, especially preferably 0.2-1.5% by weight, extremely preferably 0.5-1.0% by weight, each based on the weight of the composition, without taking into account a propellant that might be present.

Additional preferred agents according to the invention are characterized in that they include tropolone (2-hydroxy-2,4,6-cycloheptatrienone), preferably in an amount of 0.001-0.1% by weight, based on the weight of the composition, without taking into account a propellant that might be present.

Additional preferred agents according to the invention are characterized in that they include the deodorant active ingredient triethyl citrate. Triethyl citrate is a known deodorant active ingredient which acts as an enzyme inhibitor for esterases and lipases and thus contributes toward the broad band effect of the agents according to the invention. Preferred agents according to the invention include 0.5-15% by weight, preferably 3-8% by weight, extremely preferably 4-6% by weight, each based on the weight of the composition, without taking into account a propellant that might be present.

Apocrine perspiration is a complex mixture including sebum and other fats as well as steroids, among other things. Steroids themselves are not water soluble. To be transported away with the body fluids, these substances are normally in the form of sulfate or glucuronide. On the skin these steroid esters are cleaved into the volatile free steroids by hydrolytic enzymes of the skin bacteria, in particular the coryneform bacteria. In principle all bacterial exoesterases are capable of inhibiting the arylsulfatase or β-glucuronidase and therefore are preferred deodorant active ingredients according to the invention, but the enzymes arylsulfatase and β-glucuronidase are particularly effective. Compounds that inhibit arylsulfatase or β-glucuronidase are therefore preferred deodorant active ingredients according to the invention.

The development of the short-chain and medium-chain fatty acids which make a significant contribution to body odor, begins with the cleavage of the skin lipids to branched long-chain fatty acids. Cleavage of the skin lipids, which are present primarily as glycerol esters, are cleaved essentially by Propionibacterium, Corynebacterium A and Staphylococcus species (A. G. James et al., Generation and Turnover of Volatile Fatty Acids by Axillary Bacteria, 22^{nd} IFSCC Congress, Edinburgh, 2002, Poster 108). A. G. James et al have also described the fact that both short-chain C₂-C₅ fatty acids and medium-chain C₆-C₁₂ fatty acids, which are the ones mainly responsible for axillary body odor, are all formed from the long-chain branched fatty acids by the hydrolytic enzymes of a certain Corynebacterium, which A. G. James et al. call Corynebacterium A. In principle, all bacterial exoesterases are capable of this cleavage of lipid, but the enzyme lipase is particularly capable. Compounds that inhibit lipase are therefore also preferred deodorant active ingredients according to the invention.

Another class of compounds also formed in the bacterial decomposition of the constituents of perspiration and contributing to body odor are saturated and unsaturated aldehydes, especially those with a chain length of C₆-C₁₂, in particular hexanal, heptanal, octenal and nonenal. These are formed by β-cleavage of the hydroperoxides which are formed under the influence of 5-lipoxygenase on unsaturated fatty aids. Compounds that inhibit the 5-lipoxygenase enzyme are therefore preferred deodorant active ingredients according to the invention.

In addition, it is known that volatile sulfur compounds (VSC), released by enzymatic reaction in particular, are extremely foul-smelling components of human body odor and mouth odor. Sulfur-including compounds occur as water-soluble amino acid conjugates with perspiration on human skin, where they are released by skin bacteria (mainly staphylococci and corynebacteria) by enzymatic reaction. Cystathionine β-lyase is an enzyme that plays a special role in the release of VSCs. This enzyme cleaves VSCs from the amino acids and is thus an important cause in the formation of body odor. Compounds that inhibit the enzyme cystathionine β-lyase are therefore also preferred deodorant active ingredients according to the invention.

Additional preferred agents according to the invention are characterized in that they include at least one compound that is an inhibitor of the arylsulfatase enzyme. Preferred deodorant active ingredients according to the invention that act as arylsulfatase inhibitors include those disclosed in U.S. Pat. No. 5,643,559, U.S. Pat. No. 5,676,937, WO 2001/099376 A2, EP 1430879 A1 and DE 10216368 Al. Other particularly preferred agents according to the invention are characterized in that they include at least one compound, which is an inhibitor of the arylsulfatase enzyme, in a total amount of 0.001-10% by weight, preferably 0.01-5% by weight, especially preferably 0.1-2.5% by weight, each based on the weight of the composition, without taking into account a propellant that might be present.

Additional preferred agents according to the invention are characterized in that they include at least one compound that is an inhibitor of the β-glucuronidase enzyme. Preferred deodorant active ingredients according to the invention that act as β-glucuronidase inhibitors include those disclosed in WO 2003/039505 A2. Other particularly preferred agents according to the invention are characterized in that they include at least one compound, which is an inhibitor of the β-glucuronidase enzyme in a total amount of 0.001-10% by weight, preferably 0.01-5% by weight, especially preferably 0.1-2.5% by weight, each based on the weight of the composition, without taking into account a propellant that might be present.

Additional preferred agents according to the invention are characterized in that they include at least one compound that is an inhibitor of the lipase enzyme. Preferred deodorant active ingredients according to the invention that act as lipase inhibitors include those disclosed in EP 1428520 A2, additionally selected from the aminomethylene malonic acid derivatives according to DE 3018132 A1, the ethylene oxide-propylene oxide copolymers according to GB 2335596 A1 and the salts of phytic acid according to EP 650 720 Al. Other particularly preferred agents according to the invention are characterized in that they include at least one compound, which is an inhibitor of the lipase enzyme in a total amount of 0.001-10% by weight, preferably 0.01-5% by weight, especially preferably 0.1-2.5% by weight, each based on the weight of the composition, without taking into account a propellant that might be present.

Additional preferred agents according to the invention are characterized in that they include at least one compound that is an inhibitor of the 5-lipoxygenase enzyme. Preferred deodorant active ingredients according to the invention that act as 5-lipoxygenase inhibitors include those disclosed in EP 1428519 A2. Other particularly preferred agents according to the invention are characterized in that they include at least one compound, which is an inhibitor of the 5-lipoxygenase enzyme in a total amount of 0.001-10% by weight, preferably 0.01-5% by weight, especially preferably 0.1-2.5% by weight, each based on the weight of the composition, without taking into account a propellant that might be present.

Additional preferred agents according to the invention are characterized in that they include at least one compound that is an inhibitor of the cystathionine β-lyase enzyme. Preferred deodorant active ingredients according to the invention that act as cystathionine β-lyase inhibitors include those disclosed in EP 495918 B1, WO 2006/079934, DE 102010000746 Al, WO 2010/031657 A1 and WO 2010/046291 A1. Other particularly preferred agents according to the invention are characterized in that they include at least one compound, which is an inhibitor of the β-glucuronidase enzyme in a total amount of 0.001-10% by weight, preferably 0.01-5% by weight, especially preferably 0.1-2.5% by weight, each based on the weight of the composition, without taking into account a propellant that might be present.

Additional preferred agents according to the invention are characterized in that they include at least one cationic phospholipid of the formula KPL:

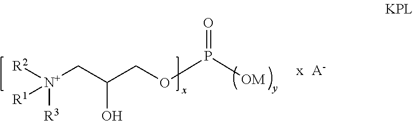

where R¹ is an alkyl, alkenyl or hydroxyalkyl group with 8 to 22 carbon atoms or an acylaminoalkyl group of the formula R⁵CONH(CₘH₂ₘ), wherein R⁵CO is a linear acyl group with 8 to 22 carbon atoms, and m=2 or 3, R² and R³ are alkyl groups with 1 to 4 carbon atoms or hydroxyalkyl groups with 2 to 4 carbon atoms or carboxyalkyl groups of the formula —(CH₂)_{z}—COOM, where z has a value of 1 to 3 and M is hydrogen or an alkali metal cation, x has a value of 1 to 3 and y has a value of (3−x), M is hydrogen or an alkali metal cation and A⁻ is an anion.

Preferred alkyl groups with 8 to 22 carbon atoms are selected from an n-octyl, n-nonyl, n-decyl, n-undecyl, lauryl, n-tridecanyl, myristyl, n-pentadecanyl, cetyl, palmityl, stearyl, elaidyl, arachidyl, behenyl and a cocyl group. A representative cocyl group consists of, based on its total weight, 4-9% by weight n-octyl, 4-9% by weight n-decyl, 45-55% by weight lauryl, 15-21% by weight myristyl, 8-13% by weight palmityl and 7-14% by weight stearyl groups. Preferred alkenyl groups with 8 to 22 carbon atoms are selected from a linoleyl group ((9Z,12Z)-octadeca-9,12-dien-1-yl) and a linolenyl group ((9Z,12Z,15Z)-octadeca-9,12,15-trien-1-yl). A preferred hydroxyalkyl group with 8 to 22 carbon atoms is selected from a 12-hydroxystearyl group.

Especially preferred cationic phospholipids of the formula KPL are those in which R¹ is an acylaminoalkyl group of the formula R⁵CONH(CₘH₂ₘ), where R⁵CO is a linear acyl group with 8 to 22 carbon atoms and m=3.

Preferred linear acyl groups R⁵CO with 8 to 22 carbon atoms are selected from an n-octanoyl, n-nonanoyl, n-decanoyl, n-undecanoyl, lauroyl, n-tridecanoyl, myristoyl, n-pentadecanoyl, cetoyl, palmitoyl, stearoyl, elaidoyl, arachidoyl, behenoyl and a cocoyl group. A representative of the cocoyl group consists of, based on its total weight 4-9% by weight n-octanoyl, 4-9% by weight n-decanoyl, 45-55% by weight lauroyl, 15-21% by weight myristoyl, 8-13% by weight palmitoyl and 7-14% by weight stearyl groups. Especially preferred linear acyl groups R⁵CO are selected from a cocoyl group, a lauroyl group (n-C₁₁H₂₃CO), a myristoyl group (n-C₁₃H₂₇CO) and a linoleoyl group ((9Z,12Z)-octadeca-9,12-dien-1-oyl). Extremely preferred linear acyl groups R⁵CO are selected from a cocoyl group, a lauroyl group (n-C₁₁H₂₃CO) and a myristoyl group (n-C₁₃H₂₇CO).

Preferred alkyl groups with 1 to 4 carbon atoms include a methyl, ethyl, n-propyl, 1-methylethyl, n-butyl, 2-methylpropyl and tert-butyl group. The methyl group is especially preferred. Preferred hydroxyalkyl group with 2 to 4 carbon atoms are a 2-hydroxyethyl group and a 1-hydroxyethyl group.

Preferred carboxyalkyl groups of the formula —(CH₂)_{z}—COOM, where z=1 to 3, include a carboxymethyl group, a carboxyethyl group and a carboxy-n-propyl group.

Preferred alkali metal cations are selected from sodium and potassium cations; Na⁺ is especially preferred. Preferred anions are selected from sulfate, chloride, phosphate, nitrate, bicarbonate and acetate, where a chloride anion is especially preferred.

Preferred agents according to the invention include a cationic phospholipid of the formula KPL as the deodorant active ingredient:

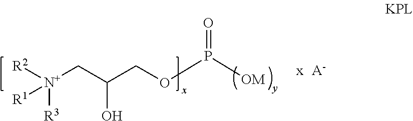

where R¹ is an acylaminoalkyl group of the formula R⁵CONH(CₘH₂ₘ)—, where R⁵CO is selected from a cocoyl group, a lauroyl group, a myristoyl group and a linoleoyl group and m=3, R² and R³ are methyl groups, x=2, y=1, M is a sodium ion and A⁻ is a chloride ion. Preferably at least one cationic phospholipid of the formula KPL having the aforementioned features is present in a total amount of 0.05-2% by weight, preferably 0.1-1% by weight, especially preferably 0.15-0.4% by weight, each based on the weight of the agent, without taking into account a propellant that might be present.

Especially preferred agents according to the invention include a cationic phospholipid of the formula KPL:

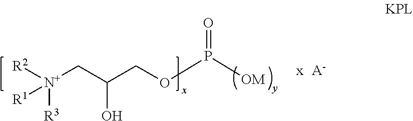

where R¹ is a cocoylaminopropyl group (also referred to as cocamidopropyl group), R² and R³ are methyl groups, x=2, y=1, M is a sodium ion and A⁻ is a chloride ion and it is obtainable under the INCI designation cocamidopropyl PG dimonium chloride phosphate in a total amount of 0.05-2% by weight, preferably 0.1-1% by weight, especially preferably 0.15-0.4% by weight, each based on the weight of the agent, without taking into account a propellant that might be present.

Additional especially preferred agents according to the invention include a cationic phospholipid of the formula KPL:

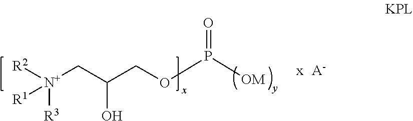

where R¹ is a myristoylaminopropyl group, R² and R³ are methyl groups, x=2, y=1, M is a sodium ion and A⁻ is a chloride ion and it is obtainable under the INCI designation myristamidopropyl PG dimonium chloride phosphate in a total amount of 0.05-2% by weight, preferably 0.1-1% by weight, especially preferably 0.15-0.4% by weight, each based on the weight of the agent, without taking into account a propellant that might be present.

Additional especially preferred agents according to the invention include a cationic phospholipid of the formula KPL:

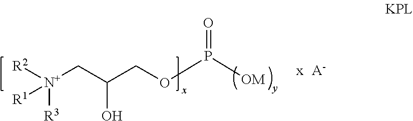

where R¹ is a lauroylaminopropyl group, R² and R³ are methyl groups, x=2, y=1, M is a sodium ion and A⁻ is a chloride ion in a total amount of 0.05-2% by weight, preferably 0.1-1% by weight, especially preferably 0.15-0.4% by weight, each based on the weight of the agent, without taking into account a propellant that might be present.

Additional deodorant active ingredients that are preferred according to the invention are odor absorbers, ion exchangers with a deodorant effect, antimicrobial agents, prebiotic components and enzyme inhibitors or, especially preferred, combinations of the aforementioned active ingredients.

Silicates are used as odor absorbers which at the same time also advantageously support the rheological properties of the composition according to the invention. The silicates that are especially preferred according to the invention include in particular sheet silicates and, of these, in particular montmorillonite, kaolinite, illite, beidellite, nontronite, saponite, hectorite, bentonite, smectite and talc. Additional preferred odor absorbers include, for example, zeolites, zinc ricinoleate, cyclodextrins, certain metal oxides such as aluminum oxide and chlorophyll. These are preferably present in a total amount of 0.1-10% by weight, especially preferably 0.5-7% by weight and extremely preferably 1-5% by weight, each based on the total composition not including propellant.

Bacteriostatic active ingredients are understood according to the invention to be active ingredients which reduce the number of skin microorganisms involved in the production of odor and/or inhibiting their growth. These microorganisms include among other various species from the group of staphylococci, from the group of corynebacteria, anaerococci and micrococci.

Bacteriostatic active ingredients that are preferred according to the invention include in particular organohalogen compounds and organohalides, quaternary ammonium compounds, a number of plant extracts and zinc compounds. These include, among others, triclosan, chlorhexidine and chlorhexidine gluconate, 3,4,4′-trichlorocarbanilide, bromochlorophen, dichlorphen, chlorothymol, chloroxylenol, hexachlorophene, dichloro-m-xylenol, dequalinium chloride, domiphen bromide ammonium phenol sulfonate, benzalkonoium halogenide, benzalkonium cetyl phosphate, benzalkonium saccharide, benzethonium chloride, cetyl pyridinium chloride, lauryl pyridinium chloride, lauryl isoquinolinium bromide, methyl benzethonium chloride. In addition, preferred deodorant active ingredients also include phenol, phenoxyethanol, disodium dihydroxyethyl sulfosuccinyl undecylenate, sodium bicarbonate, zinc lactate, sodium phenol sulfonate and zinc phenol sulfonate, ketoglutaric acid, terpene alcohols such as, for example, farnesol, chlorophyllin copper complexes, α-monoalkyl glycerol ethers with a branched or linear saturated or unsaturated optionally hydroxylated C₆-C₂₂ alkyl radical, especially preferably α-(2-ethylhexyl)glycerol ether available commercially as Sensiva® SC 50 (from Schülke & Mayr) as well as carboxylic acid esters of mono-, di- and triglycerol (e.g., glycerol monolaurate, diglycerol monocaprinate).

Preferred antiperspirant agents according to the invention include at least one deodorant active ingredient selected from silver salts, aromatic alcohols of the structure AA-1 with the aforementioned substituents, 1,2-alkanediols with 5 to 12 carbon atoms, α-(2-ethylhexyl)glycerol ether (3-(2-ethylhexyloxy)-1,2-propanediol), tropolone, triethyl citrate, cationic phospholipids of the formula KPL with the aforementioned substituents as well as mixtures thereof.

Additional preferred antiperspirant compositions according to the invention are characterized in that the at least one deodorant active ingredient is present in a total amount of 0.2-20% by weight, preferably 1-15% by weight, especially preferably 1.5-5% by weight, wherein the amounts in percent by weight are based on the total weight of the composition, without taking into account a propellant that might be present.

In another especially preferred embodiment, the compositions according to the invention include at least one deodorant active ingredient in a total amount of 0.0001-40% by weight, preferably 0.2-20% by weight, especially preferably 1-15% by weight, extremely preferably 1.5-5% by weight as well as at least one antiperspirant aluminum salt in a total amount of 2-40% by weight, preferably 8-35% by weight, especially preferably 10-28% by weight and extremely preferably 12-20% by weight, wherein the amounts in percent by weight are based on the total weight of the active substance (USP), which is free of ligands and free of water or crystallization in the composition, without taking into account a propellant that might be present.

Preferred agents according to the invention preferably also include at least one cosmetic oil that is liquid under standard conditions and is not a perfume or an essential oil.

The cosmetic oil is liquid under standard conditions. Essential oils are understood according to the invention to be mixtures of volatile components produced by steam distillation from plant-based raw materials such as citrus oils.

The total amount of cosmetic oils that are liquid under standard conditions and are not a perfume and not an essential oil amounts to 1-95% by weight, preferably 5-90% by weight, especially preferably 30-75% by weight, extremely preferably 50-60% by weight in preferred compositions according to the invention where the amounts indicated are based on the weight of the composition, without taking into account a propellant that might be present.

Additional agents preferred according to the invention include at least one cosmetic oil which is not a perfume and is not an essential oil, in a total amount of 1-85% by weight, preferably 5-50% by weight, especially preferably 9-25% by weight, extremely preferably 15-20% by weight, each based on the total weight without taking into account a propellant that might be present.

With the cosmetic oils a distinction is made between volatile oils and nonvolatile oils. Nonvolatile oils are understood to be oils that have a vapor pressure of less than 2.66 Pa (0.02 mmHg) at 20° C. and an ambient pressure of 1013 hPa. Volatile oils are understood to be oils that have a vapor pressure of 2.66 Pa to 40,000 Pa (0.02-300 mmHg), preferably 10-12,000 Pa (0.1-90 mmHg), especially preferably 13-3000 Pa, extremely preferably 15-500 Pa at 20° C. and an ambient pressure of 1013 hPa.

Volatile cosmetic oils are usually selected from cyclic silicone oils with the INCI designations cyclomethicone. The INCI designation cyclomethicone is understood to include in particular cyclotrisiloxane (hexamethylcyclotrisiloxane), cyclotetrasiloxane (octamethylcyclotetrasiloxane), cyclopentasiloxane (decamethylcyclopentasiloxane) and cyclohexasiloxane (dodecamethylcyclohexasiloxane). These oils have a vapor pressure of approx. 13-15 Pa at 20° C.

Cyclomethicones are known in the prior art to be very suitable oils for cosmetic compositions, in particular for deodorant compositions such as sprays and sticks. However, it may be preferable according to the invention to refrain from using cyclomethicones because of their persistence in the environment. In an especially preferred embodiment, the compositions according to the invention and the compositions used according to the invention include 0 to less than 1% by weight, preferably max. 0.1% by weight cyclomethicone, based on the weight of the composition, wherein any propellant that might be present is not taken into account.

A cyclomethicone substitute that is preferred according to the invention is a mixture of C₁₃-C₁₆ isoparaffins, C₁₂-C₁₄ isoparaffins and C₁₃-C₁₅ alkanes, which have a viscosity at 25° C. in the range of 2-6 mPas and which have a vapor pressure of 20° C. in the range of 10-150 Pa, preferably 100-150 Pa. Such a mixture is available, for example, under the designation SiClone SR-5 from the company Presperse Inc.

Additional preferred volatile silicone oils are selected from volatile linear silicone oils in particular volatile linear silicone oils with 2 to 10 siloxane units, such as hexamethyldisiloxane (L₂), octamethyltrisiloxane (L₃), decamethyltetrasiloxane (L₄), such as those included in the commercial products DC2-1184, Dow Corning® 200 (0.65 cSt) and Dow Corning® 200 (1.5 cSt) from Dow Corning, for example, and low-molecular phenyl trimethicone with a vapor pressure of approx. 2000 Pa at 20° C., such as that available under the name Baysilone Fluid PD 5 from GE Bayer Silicones/Momentive, for example.

Preferred antiperspirant compositions according to the invention include at least one volatile silicone oil, which may be cyclic or linear, because of the drier skin feel and faster release of the active ingredient.

Additional preferred compositions according to the invention include at least one volatile non-silicone oil because of the drier feeling on the skin and the more rapid release of the antiperspirant active ingredient. Preferred volatile non-silicone oils are selected from C₈-C₁₆ isoparaffins in particular isononane, isodecane, isoundecane, isododecane, isotridecane, isotetradecane, isopentadecane and isohexadecane as well as mixtures thereof. C₁₀-C₁₃ isoparaffin mixtures in particular those with a vapor pressure of 10-400 Pa, preferably 13-100 Pa at 20° C. are preferred.

This at least one C₈-C₁₆ isoparaffin is preferably present in a total amount of 1-60% by weight, preferably 3-45% by weight, especially preferably 5-40% by weight, extremely preferably 8-20% by weight, each based on the total weight of the composition, without taking into account a propellant that might be present.

Additional compositions that are preferred according to the invention include at least one nonvolatile cosmetic oil, selected from nonvolatile silicone oils and nonvolatile non-silicone oils. Residues of insoluble ingredients in the composition such as talc, but also the antiperspirant active ingredients that dry on the skin (=antiperspirant aluminum salts), can be masked successfully with a nonvolatile oil. Furthermore, parameters such as skin feel, visibility of the residue and stability of the compositions according to the invention can be finely regulated with a mixture of various oils, in particular a mixture of volatile and nonvolatile oils, and these parameters can be adapted better to the needs of the consumer.

Agents that are preferred according to the invention are characterized in that the cosmetic oil that does not include any perfume or any essential oil includes at least one volatile oil with a vapor pressure of 10-3000 Pa at 20° C., which is not a perfume and not an essential oil, in a total amount of 10-100% by weight, especially preferably 30-80% by weight, each based on the total weight of the cosmetic oils.

It is of course also possible to formulate agents according to the invention with a small amount of volatile oils—in other words, with 0.5-15% by weight of volatile oils, based on the total weight of the agent, without taking a propellant into account—or even without volatile oils.

Especially preferred oils according to the invention are esters of the linear or branched saturated or unsaturated fatty alcohols with 2 to 30 carbon atoms with linear or branched, saturated or unsaturated fatty acids with 2 to 30 carbon atoms, which may be hydrolyzed. It should be pointed out here that some esters of linear or branched C₁-C₂₂ alkanols or C₁₄-C₂₂ alkenols and some triesters of glycerol with linear or branched C₂-C₂₂ carboxylic acids that may be saturated or unsaturated are solid under standard conditions, such as cetyl stearate or glycerol tristearate (=stearin), for example. These esters, which are solid under standard conditions, are not cosmetic oils according to the invention because they do not satisfy the condition “liquid under standard conditions.” It is within the scope of general knowledge of those skilled in the art to ascertain whether such an ester is liquid or solid under standard conditions.

Esters of linear or branched saturated fatty alcohols with 2 to 5 carbon atoms with linear or branched saturated or unsaturated fatty acid with 3 to 18 carbon atoms, which may be hydroxylated are preferred. Preferred examples of these include diisopropyl palmitate, isopropyl stearate, isopropyl myristate, 2-hexyldecyl stearate, 2-hexyldecyl laurate, isodecyl neopentanoate, isononyl isononanoate, 2-ethylhexyl palmitate and 2-ethylhexylstearate. Also preferred are isopropyl isostearate, isopropyl oleate, isooctyl stearate, isononyl stearate, isocetyl stearate, isononyl isononanoate, isotridecyl isononanoate, cetearyl isononanoate, 2 ethylhexyl laurate, 2-ethylhexyl isostearate, 2-ethylhexyl cocoate, 2-octyldodecyl palmitate, butyl octanoic acid 2-butyl octanoate, diisotridecyl acetate, n-butylstearate, n-hexyl laurate, n-decyl oleate, oleyl oleate, oleyl erucate, erucyl oleate, erucyl erucate, ethylene glycol dioleate, ethylene glycol dipalmitate, n-hexyl laurate, n-decyl oleate, oleyl oleate, oleyl erucate, erucyl oleate, C₁₂-C₁₅-alkyl lactate and di-C₁₂-C₁₃-alkyl malate as well as the benzoic acid esters of linear or branched C₈-22 alkanols. Especially preferred are benzoic acid C₁₂-C₁₅-alkyl esters, which can be obtained, for example, as the commercial product Finsolv® TN (C₁₂-C₁₅ alkyl benzoate) as well as benzoic acid isostearyl esters, obtainable, for example, as Finsolv® SB, 2-ethylhexyl benzoate, obtainable, for example, as Finsolv® EB and benzoic acid 2-octyldodecyl ester, obtainable, for example, as Finsolv® BOD.

Other oil components that are preferred according to the invention are selected from the C₈-C₂₂ fatty alcohol esters of monovalent or polyvalent C₂-C₇ hydroxycarboxylic acids, in particular the esters of glycolic acid, lactic acid, malic acid, tartaric acid, citric acid and salicylic acid. Such esters based on linear C₁₄₁₁₅ alkanols, for example, C₁₂-C₁₅ alkyl lactate and C_{12/13} alkanols branched in position 2 are available under the brand name Cosmacol® from the company Nordmann, Rassmann GmbH & Co., Hamburg, in particular the commercial products Cosmacol® ESI, Cosmacol® EMI and Cosmacol® ETI.

Use of isopropyl esters of C₁₂-C₁₈ carboxylic acids, in particular the use of isopropyl myristate and especially preferably mixtures of isopropyl myristate with C₁₀-C₁₃ isoparaffin mixtures have proven to be especially advantageous, for example, for the release of active ingredients, the latter preferably with a vapor pressure of 10-400 Pa at 20° C.

Preferred agents according to the invention include at least one ester of the linear or branched saturated or unsaturated fatty alcohols with 2 to 30 carbon atoms with linear or branched saturated or unsaturated fatty acids with 2 to 30 carbon atoms, which may be hydroxylated, in a total amount of 1-80% by weight, preferably 5-26% by weight, especially preferably 9-24% by weight, extremely preferably 12-17% by weight, each based on the weight of the total composition, without taking into account a propellant that might be present.

Another especially preferred ester oil is triethyl citrate. Additional preferred products according to the invention include triethyl citrate and at least one C₈-C₁₆ isoparaffin selected from isononane, isodecane, isoundecane, isododecane, isotridecane, isotetradecane, isopentadecane and isohexadecane as well as mixtures of these isoparaffins. Additional preferred products according to the invention include triethyl citrate and at least one C₈-C₁₆ isoparaffin selected from isononane, isodecane, isoundecane, isododecane, isotridecane as well as mixtures of these C₈-C₁₆ isoparaffins. Additional products preferred according to the invention include triethyl citrate and a mixture of isodecane, isoundecane, isododecane and isotridecane.

Additional non-volatile, non-silicone oils that are preferred according to the invention are selected from branched, saturated or unsaturated fatty alcohols with 6 to 30 carbon atoms. These alcohols are often also referred to as Guerbet alcohols because they are obtainable according to the Guerbet reaction. Preferred alcohol oils are 2-hexyldecanol, 2-octyldodecanol and 2-ethylhexyl alcohol. Also preferred is isostearyl alcohol. Additional preferred nonvolatile oils are selected from mixtures of Guerbet alcohols and Guerbet alcohol esters, e.g., 2-hexyldecanol and 2-hexyldecyl laurate.

The term “triglyceride” as used below is understood to mean “glycerol triester.” Other non-volatile oils that are preferred according to the invention are selected from the triglycerides of linear or branched saturated or unsaturated optionally hydroxylated C₈₋₃₀ fatty acids if they are liquid under standard conditions. The use of natural oils, e.g., soy oil, cottonseed oil, sunflower oil, palm oil, palm kernel oil, linseed oil, almond oil, castor oil, corn oil, canola oil, olive oil, sesame oil, thistle oil, wheat germ oil, peach kernel oil and the liquid fractions of coconut oil and the like may be especially suitable. Especially preferred are the synthetic triglyceride oils, in particular capric/caprylic triglycerides, e.g., the commercial products Myritol® 318 or Myritol® 331 (BASF/BASF) with unbranched fatty acid radicals as well as glyceryl triisostearin and glyceryl tri(2-ethylhexanoate) with branched fatty acid radicals. Such triglyceride oils preferably constitute an amount of less than 50% by weight of the total weight of all cosmetic oils in the composition according to the invention. Especially preferably the total weight of triglyceride oils amounts to 0.5-25% by weight, preferably 1-5% by weight, each based on the total composition, without taking into account a propellant that might be present.

Additional nonvolatile non-silicone oils that are especially preferred according to the invention are selected from the dicarboxylic acid esters of linear or branched C₂-C₁₀ alkanols, in particular diisopropyl adipate, di-n-butyl adipate, di-(2-ethylhexyl) adipate, dioctyl adipate, diethyl/di-n-butyl/dioctyl sebacate, diisopropyl sebacate, dioctyl malate, dioctyl maleate, dicaprylyl maleate, diisooctyl succinate, di-2-ethylhexyl succinate and di-(2-hexyldecyl) succinate.

Additional non-volatile non-silicone oils that are especially preferred according to the invention are selected from the addition products of 1 to 5 propylene oxide units onto monovalent or polyvalent C₈₋₂₂ alkanols, such as octanol, decanol, decanediol, lauryl alcohol, myristyl alcohol and stearyl alcohol, e.g., PPG-2 myristyl ether and PPG-3 myristyl ether.

Additional non-volatile non-silicone oils that are especially preferred according to the invention are selected from the addition products of at least 6 ethylene oxide and/or propylene oxide units onto monovalent or polyvalent C₃₋₂₂ alkanols such as glycerol, butanol, butanediol, myristyl alcohol and stearyl alcohol, which may be esterified if desired, e.g., PPG-14 butyl ether, PPG-9 butyl ether, PPG-10 butanediol and PPG-15 stearyl ether.

Additional non-volatile non-silicone oils that are especially preferred according to the invention are selected from the symmetrical, asymmetrical or cyclic esters of carbonic acid with C₆-C₂₀ alcohols, e.g., di-n-caprylyl carbonate (Cetiol® CC) or di-(2-ethylhexyl) carbonate (Tegosoft DEC). However, the esters of carbonic acid with C₁-C₅ alcohols, e.g., glycerol carbonate or propylene carbonate, are not compounds that are suitable as a cosmetic oil.

Additional oils that may be preferred according to the invention are selected from the esters of dimers of unsaturated C₁₂-C₂₂ fatty acids (dimer fatty acids) with monovalent linear branched or cyclic C₂-C₁₈ alkanols or with polyvalent linear or branched C₂-C₆ alkanols. The total weight of dimer fatty acid esters especially preferably amounts to 0.5-10% by weight, preferably 1-5% by weight, each based on the total composition, without taking into account a propellant that might be present.

Additional cosmetic oils that are especially preferred according to the invention are selected from nonvolatile silicone oils. Preferred non-volatile silicone oils according to the invention are selected from linear polyalkylsiloxanes with a kinematic viscosity at 25° C. of at least 5 cSt to 2000 cSt, in particular selected from linear dimethylsiloxanes with a kinematic viscosity of 5 cSt to 2000 cSt, preferably 10 to 350 cSt, especially preferably 50-100 cSt at 25° C., such as those available, for example, under the brand names Dow Corning 200 and/or Xiameter PMX from Dow Corning and/or Xiameter. Other preferred nonvolatile silicone oils include phenyl trimethicone with a kinematic viscosity of 10 to 100 cSt at 25° C., preferably 15 to 30 cSt and cetyldimethicone.

Natural and synthetic hydrocarbons that are preferred according to the invention are selected from paraffin oils, isohexadecane, isoeicosane, polyisobutenes and polydecenes, which are obtainable under the brand name Emery® 3004, 3006, 3010 or under the brand name Nexbase® 2004G from Nestle as well as 1,3-di-(2-ethylhexyl)cyclohexane.

The compositions according to the invention and those used according to the invention optionally include additional vehicles, excipients and additives.

Aerosol sprays and pump sprays may be present as a suspension, water-in-oil emulsion, oil-in-water emulsion, silicon oil-in-water emulsion, water-in-oil microemulsion, oil-in-water microemulsion, silicone oil-in-water microemulsion, alcoholic solution, in particular ethanolic solution, hydroalcoholic solution, in particular solutions with more than 50% by weight of a water-ethanol mixture, glycolic solution, in particular as a solution in 1,2-propylene glycol, glycerol, dipropylene glycol and liquid (under standard conditions) polyethylene glycols, hydroglycolic solution, polyol solution and as water-polyol solution. All the aforementioned compositions may be thickened, for example, based on fatty acid soaps, dibenzylidene sorbitol, N-acylamino acid amides, 12-hydroxystearic acid, polyacrylates of the carbomer and carbopol types, polyacrylamides and polysaccharides, which may be chemically and/or physically modified, as well as in particular by sheet silicates, especially preferably by montmorillonite, kaolinite, illite, beidellite, nontronite, saponite, hectorite, bentonite, smectite and talc. The agents may be transparent, translucent or opaque.

The agents according to the invention may preferably include at least one lipid or wax component with a melting point of >50° C. as long as it is not present in such large amounts that the agents can no longer be sprayed. A corresponding total amount is preferably 0.001-5% by weight, especially preferably 0.01-2% by weight, each based on the weight of the total agent, without taking into account a propellant that might be present.

Natural vegetable waxes are preferred according to the invention, for example, candelilla wax, carnauba wax, japan wax, sugar cane wax, ouricuri wax, cork wax, sunflower wax, fruit waxes such as orange waxes, lemon waxes, grapefruit wax and animal waxes, for example, beeswax, shellac wax and spermaceti. In the sense of the invention it may be especially preferable to use hydrogenated or hardened waxes. The wax components may also be chemically modified waxes, in particular the hard waxes such as montan ester waxes, hydrogenated jojoba waxes and sasol waxes. The synthetic waxes which are also preferred according to the invention include, for example, polyalkylene waxes and polyethylene glycol waxes, C₂₀-C₄₀ dialkyl esters of dimer acids, C₃₀₋₅₀ alkyl beeswax and alkyl and alkylaryl esters of dimer fatty acids.

An especially preferred wax component is selected from at least one ester of a saturated monovalent C₁₆-C₆₀ alcohol and a saturated C₈-C₃₆ monocarboxylic acid. According to the invention these also includes lactides, the cyclic double esters of α-hydroxycarboxylic acids of the corresponding chain length. Esters of fatty acids and long-chain alcohols have proven to be especially advantageous for the composition according to the invention because they impart excellent sensorial properties to the antiperspirant preparation and impart a high stability to the stick on the whole. The esters are composed of saturated branched or unbranched monocarboxylic acids and saturated branched or unbranched monohydric alcohols. Esters of aromatic carboxylic acids and/or hydroxycarboxylic acids (for example, 12-hydroxystearic acid) and saturated branched or unbranched alcohols can also be used according to the invention if the wax component has a melting point >50° C. It is especially preferable to select the wax components from the group of esters of saturated branched or unbranched alkane carboxylic acids with a chain length of 12 to 24 carbon atoms and the saturated branched or unbranched alcohols with a chain length of 16 to 50 carbon atoms and a melting point >50° C. In particular the wax component may advantageously be C₁₆₋₃₆ alkyl stearates and C₁₈₋₃₈ alkylhydroxystearoyl stearates, C₂₀₋₄₀ alkyl erucates and cetearyl behenate. The wax or the wax components have a melting point >50° C., preferably >60° C. An especially preferred embodiment of the invention includes as the wax component a C₂₀-C₄₀ alkyl stearate. Another especially preferred embodiment of the invention includes as the wax component cetearyl behenate, i.e., mixtures of cetyl behenate and stearyl behenate.

Additional preferred lipid or wax components with a melting point >50° C. include the triglycerides of saturated and optionally hydroxylated C₁₂₋₃₀ fatty acids such as hardened triglyceride fats (hydrogenated palm oil, hydrogenated coconut oil, hydrogenated castor oil), glyceryl tribehenate (tribehenin) or glyceryl tri-12-hydroxysteareate, also synthetic full esters of fatty acids and glycols or polyols with 2-6 carbon atoms as long as they have a melting point above 50° C., for example, preferably C₁₈-C₃₆ acid triglyceride (Syncrowax® HGL-C). Hydrogenated castor oil obtainable, for example, as a commercial product Cutina® HR is especially preferred as the wax component according to the invention.

Additional preferred lipid or wax components with a melting point >50° C. include the saturated linear C₁₄-C₃₆ carboxylic acids, in particular myristic acid, palmitic acid, stearic acid and behenic acid as well as mixtures of these compounds.

The agents according to the invention especially preferably also include at least one emulsifier and/or at least one surfactant.

Suitable emulsifiers and surfactants that are preferred according to the invention are selected from anionic, cationic, nonionic, amphoteric in particular ampholytic and zwitterionic emulsifiers and surfactants.

Surfactants are amphiphilic (bifunctional) compounds consisting of at least one hydrophobic part of the molecule and at least one hydrophilic part of the molecule. The hydrophobic radical is preferably a hydrocarbon chain with 8 to 28 carbon atoms, which may be saturated or unsaturated, linear or branched. This C₈-C₂₈ alkyl chain is especially preferably linear.

Basic properties of the surfactants and emulsifiers are the oriented absorption on interfaces as well as the aggregation to micelles and the formation of lyotrophic phases.

Anionic surfactants are understood to be surfactants with exclusively anionic charges. They include carboxyl groups, sulfonic acid groups or sulfate groups, for example. Especially preferred anionic surfactants are alkyl sulfates, alkyl ether sulfates, acyl glutamates and C₈-C₂₄ carboxylic acids as well as their salts, the so-called soaps.

Cationic surfactants are understood to be surfactants with exclusively cationic charges. They include quaternary ammonium groups, for example. Cationic surfactants of the type of quaternary ammonium compounds, the ester quats and the amidoamines are preferred. Preferred quaternary ammonium compounds include ammonium halides as well as the imidazolium compounds known by the INCI designations quaternium-27 and quaternium-83. Other cationic surfactants that can be used according to the invention include the quaternized protein hydrolysates. Preferred ester quats are quaternized ester salts of fatty acids with triethanolamine, quaternized ester salts of fatty acids with dimethanol alkylamines and quaternized ester salts of fatty acids with 1,2-dihydroxypropyl dialkylamines. The cationic surfactants are preferably present in the preferred agents according to the invention in amounts of 0.05 to 10% by weight, based on the total agent.

The amphoteric surfactants are subdivided into ampholytic surfactants and zwitterionic surfactants. Ampholytic surfactants are understood to be surface-active compounds which have both acidic groups (for example, —COOH or —SO₃H groups) as well as basic hydrophilic groups (for example, amino groups) and which thus have either acidic or basic properties, depending on the conditions. Those skilled in the art understand zwitterionic surfactants to be surfactants having both a negative charge and a positive charge in the same molecule.

Examples of preferred zwitterionic surfactants include the betaines, the N-alkyl-N,N-dimethylammonium glycinates, the N-acylaminopropyl-N,N-dimethylammonium glycinates and the 2-alkyl-3-carboxymethyl-3-hydroxyethylimidazolines, each with 8 to 24 carbon atoms in the alkyl group.

Examples of preferred ampholytic surfactants include N-alkylglycines, N-alkylaminopropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids each with 8 to 24 carbon atoms in the alkyl group.

### Oil-in-Water Emulsifiers

The compositions according to the invention which are formulated as emulsions, in particular as oil-in-water emulsions preferably include at least one nonionic oil-in-water emulsifier with an HLB value of more than 7 to 20. These are emulsifiers with which those skilled in the art are familiar in general such as those listed for example, in Kirk-Othmer, Encyclopedia of Chemical Technology, 3^{rd} edition, 1979, vol. 8, pages 913-916. For ethoxylated products the HLB value is calculated according to the formula HLB=(100−L):5, where L is the amount by weight of the lipophilic groups, i.e., the fatty alkyl or fatty acyl groups in the ethylene oxide adducts, expressed in percent by weight.

In the choice of the nonionic oil-in-water emulsifiers suitable according to the invention, it is especially preferable to use a mixture of nonionic oil-in-water emulsifiers to be able to optimally adjust the stability of the agents according to the invention. The individual emulsifier components supply an amount to the total HLB value or average HLB value of the oil-in-water emulsifier mixture according to their quantitative proportion in the total amount of the oil-in-water emulsifiers. According to the invention the average HLB value of the oil-in-water emulsifier mixture is 10-19 preferably 12-18 and especially preferably 14-17. Oil-in-water emulsifiers from the HLB value ranges of 10-14, 14-16 and optionally 16-19 are preferably combined with each other to obtain such average HLB values. The oil-in-water emulsifier mixtures of course may also include nonionic emulsifiers with HLB values in the range of >7-10 and 19-20. Such emulsifier mixtures may also be preferred according to the invention. However, in another preferred embodiment, the antiperspirant compositions according to the invention may also include just a single oil-in-water emulsifier with an HLB value in the range of 10-19.

Preferred antiperspirant agents according to the invention include at least one nonionic oil-in-water emulsifier selected from ethoxylated C₈-C₂₄ alkanols with an average of 10-100 mol ethylene oxide per mol, ethoxylated C₈-C₂₄ carboxylic acids with an average of 10-100 mol ethylene oxide per mol, silicone copolyols with ethylene oxide unit or with ethylene oxide and propylene oxide units, alkyl mono- and oligoglycosides with 8 to 22 carbon atoms in the alkyl radical and their ethoxylated analogs, ethoxylated sterols, partial esters of polyglycerols with 2 to 10 glycerol units esterified with 1-4 saturated or unsaturated linear or branched optionally hydroxylated C₈-C₃₀ fatty acid radicals if they have an HLB value of more than 7 as well as mixtures of the aforementioned substances.

The ethoxylated C₈-C₂₄ alkanols have the formula R¹O(CH₂CH₂O)ₙH, where R¹ stands for a linear or branched alkyl or alkenyl radical with 8 to 24 carbon atoms and n stands for the average number of ethylene oxide units per molecule, for numbers of 10-100, preferably 10-30 mol ethylene oxide on 1 mol capryl alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol as well as industrial mixtures thereof. Adducts of 10-100 mol ethylene oxide onto industrial fatty alcohols with 12 to 18 carbon atoms such as coconut, palm, palm kernel or tallow fatty alcohol are also suitable.

The ethoxylated C₈-C₂₄ carboxylic acids have the formula R¹(OCH₂CH₂)ₙ where R¹ stands for a linear or branched saturated or unsaturated acyl radical with 8 to 24 carbon atoms and n stands for the average number of ethylene oxide units per molecule, for numbers of 10-100, preferably 10-30 mol ethylene oxide per mol caprylic acid, 2-ethylhexanoic acid, capric acid, lauric acid, isotridecanoic acid, myristic acid, cetyl acid, palmitoleic acid, stearic acid, isostearic acid, oleic acid, elaidic acid, petroselinic acid, arachic acid, gadolineic acid, behenic acid, erucaic acid and brassidic acid as well as their industrial mixtures. Adducts of 10-100 mol ethylene oxide onto industrial fatty acids with 12 to 18 carbon atoms such as coconut, palm, palm kernel or tallow fatty acid are also suitable. PEG-50 monostearate, PEG-100 monostearate, PEG-50 monooleate, PEG-100 monooleate, PEG-50 monolaurate and PEG-100 monolaurate are especially preferred.

The C₁₂-C₁₈ alkanols or C₁₂-C₁₈ carboxylic acids each with 10-30 units of ethylene oxide per molecule as well as mixtures of these substances are especially preferred, in particular Ceteth-12, Ceteth-20, Ceteth-30, Steareth-12, Steareth-20, Steareth-30, Laureth-12 and Beheneth-20.

In addition C₈-C₂₂ alkyl mono- and oligoglycosides are preferably also used. C₈-C₂₂ alkyl mono- and oligoglycosides are known commercial surfactants and emulsifiers. They are produced in particular by reading glucose or oligosaccharides with primary alcohols with 8 to 22 carbon atoms. With regard to the glycoside radical it is true that both monoglycosides in which a cyclic sugar radical is glycosidically bound to the fatty alcohol as well as oligomer glycosides with a degree of oligomerization up to approx. 8, preferably 1-2 are suitable. The degree of oligomerization is a statistical average on which the usual homolog distribution for such industrial products is based. Products obtainable under the name Plantacare© include a glucosidically bound C₈-C₁₆ alkyl group on an oligoglucoside radical whose average degree of oligomerization is 1-2, in particular 1.1-1.4. Especially preferred C₈-C₂₂ alkyl mono- and oligoglycosides are selected from octyl glucoside, decyl glucoside, lauryl glucoside, palmityl glucoside, isostearyl glucoside, stearyl glucoside, arachidyl glucoside and behenyl glucoside as well as mixtures thereof. The acyl glucamides derived from glutamine are also suitable as nonionic oil-in-water emulsifiers.

Ethoxylated sterols, in particular ethoxylated soy sterols are suitable oil-in-water emulsifiers according to the invention. The degree of ethoxylation can be greater than 5, preferably at least 10, to have an HLB value greater than 7 to 20. Suitable commercial products include PEG-10 soy sterol, PEG-16 soy sterol and PEG-25 soy sterol.

Partial esters of polyglycerols with 2 to 10 glycerol units and esterified with 1 to 4 saturated or unsaturated, linear or branched, optionally hydroxylated C₈-C₃₀ fatty acid radicals are preferred for use if they have an HLB value in the range of more than 7 to 20. Especially preferred examples include diglycerol monocaprylate, diglycerol monocaprate, diglycerol monolaurate, triglycerol monocaprylate, triglycerol monocaprate, triglycerol monolaurate, tetraglycerol monocaprylate, tetraglycerol monocaprate, tetraglycerol monolaurate, pentaglycerol monocaprylate, pentaglycerol monocaprate, pentaglycerol monolaurate, hexaglycerol monocaprylate, hexaglycerol monocaprate, hexaglycerol monolaurate, hexaglycerol monomyristate, hexaglycerol monostearate, decaglycerol monocaprylate, decaglycerol monocaprate, decaglycerol monolaurate, decaglycerol monomyristate, decaglycerol monoisostearate, decaglycerol monostearate, decaglycerol monooleate, decaglycerol monohydroxystearate, decaglycerol dicaprylate, decaglycerol dicaprate, decaglycerol dilaurate, decaglycerol dimyristate, decaglycerol diisostearate, decaglycerol distearate, decaglycerol dioleate, decaglycerol dihydroxystearate, decaglycerol tricaprylate, decaglycerol tricaprate, decaglycerol trilaurate, decaglycerol trimyristate, decaglycerol triisostearate, decaglycerol tristearate, decaglycerol trioleate and decaglycerol trihydroxystearate.

Especially preferred antiperspirants according to the invention are characterized in that the nonionic oil-in-water emulsifier is included in a total amount of 0.01-10% by weight, especially preferably 0.1-4% by weight and extremely preferably 0.5-3% by weight, based on the total weight of the propellant-free agent.

### Water-in-Oil Emulsifiers

Preferred compositions according to the invention that are formulated as an emulsion preferably also include at least one nonionic water-in-oil emulsifier with an HLB value of more than 1.0 and less than or equal to 7.0, selected from the mono- and diesters of ethylene glycol and the mono-, di-, tri- and tetraesters of pentaerythritol with linear saturated fatty acids with 12 to 30, in particular 14 to 22 carbon atoms, which may be hydroxylated, as well as mixtures thereof, as agents to impart consistency and/or water binding agents. The mono- and diesters are preferred according to the invention. Preferred C₁₂-C₃₀ fatty acid radicals according to the invention are selected from lauric acid, palmitic acid, stearic acid, arachic acid and behenic acid radicals. The stearic acid radical is especially preferred. Especially preferred nonionic water-in-oil emulsifiers according to the invention with an HLB value greater than 1.0 and less than or equal to 7.0 are selected from pentaerythrityl monostearate, pentaerythrityl distearate, pentaerythrityl tristearate, pentaerythrityl tetrastearate, ethylene glycol monostearate, ethylene glycol distearate and mixtures thereof. Especially preferred water-in-oil emulsifiers with an HLB value greater than 1.0 and less than or equal to 7.0 according to the invention are available for example, as commercial products Cutina® PES pentaerythrityl distearate), Cutina® AGS (INCI: glycol distearate) or Cutina® EGMS (INCI: glycol stearate). These commercial products are already mixtures of mono- and diesters (in the case of the pentaerythritol esters, tri- and tetraesters are also included). According to the invention it may be preferable to use only a single water-in-oil emulsifier. In another preferred embodiment the compositions according to the invention include mixtures in particular industrial mixtures of at least two water-in-oil emulsifiers. An industrial mixture is understood to be, for example, a commercial product such as Cutina® PES.

In addition to the aforementioned water-in-oil emulsifiers based on ethylene glycol or pentaerythrityl esters, in a preferred embodiment at least one other nonionic water-in-oil emulsifier with an HLB value greater than 1.0 and less than or equal to 7.0 may also be present, but its proportion of the total weight of nonionic water-in-oil emulsifiers with an HLB value greater than 1.0 and less than or equal than 7.0 should preferably be no greater than 80%. In an especially preferred embodiment, the compositions according to the invention include the at least one additional water-in-oil emulsifier with an HLB value greater than 1.0 and less than or equal to 7.0 only in an amount by weight of max. 10% and/or they are free of additional water-in-oil emulsifiers. Some of these additional suitable emulsifiers are listed for example, in Kirk-Othmer, Encyclopedia of Chemical Technology, 3^{rd} edition, 1979, Vol. 8, page 913. The HLB value can also be calculated for ethoxylated adducts, as already mentioned.

The following are preferably suitable as the water-in-oil emulsifier:
linear saturated alkanols with 12 to 30 carbon atoms, in particular with 16 to 22 carbon atoms, in particular cetyl alcohol, stearyl alcohol, arachidyl alcohol, behenyl alcohol and lanolin alcohol or mixtures of these alcohols such as those available in industrial hydrogenation of vegetable and animal fatty acids,
esters and in particular partial esters of a polyol with 3 to 6 carbon atoms and linear saturated and unsaturated fatty acids with 12 to 30 carbon atoms, in particular 14 to 22 carbon atoms, which may be hydroxylated. Such esters or partial esters are for example, the mono- and diesters of glycerol or the monoesters of propylene glycol with linear saturated and unsaturated C₁₂-C₃₀ carboxylic acids that may be hydroxylated, in particular those with palmitic acid and stearic acid, the sorbitan mono-, di- or triesters of linear saturated and unsaturated C₁₂-C₃₀ carboxylic acids that may be hydroxylated, in particular those of myristic acid, palmitic acid, stearic acid or mixtures of these fatty acids and the methyl glucose mono- and diesters of linear saturated and unsaturated C₁₂-C₃₀ carboxylic acids which may be hydroxylated;
sterols, i.e., steroids which have a hydroxyl group on the C₃ atom of the steroid backbone and are isolated from animal tissue (zoosterols, e.g., cholesterol, lanosterol) as well as from plants (phytosterols, e.g., ergosterol, stigmasterol, sitosterol) and from fungi and yeasts (mycosterols) and may have a low degree of ethoxylation (1-5 EO);
alkanols and carboxylic acids, each with 8 to 24 carbon atoms, in particular with 16 to 22 carbon atoms in the alkyl group and 1-4 ethylene oxide units per molecule, having an HLB value greater than 1.0 and less than or equal to 7.0,
glycerol monoethers of saturated and/or unsaturated branched and/or unbranched alcohols with a chain length of 8 to 30, in particular 12 to 18 carbon atoms,
partial esters of polyglycerols with n=2 to 10 glycerol units and esterified with 1 to 5 saturated or unsaturated, linear or branched optionally hydroxylated C₈-C₃₀ fatty acid radicals if they have an HLB value of less than or equal to 7,
as well as mixtures of the aforementioned substances.

According to invention it may be preferable to use only one single additional water-in-oil emulsifier. In another preferred embodiment the compositions according to the invention include mixtures, in particular industrial mixtures of at least two additional water-in-oil emulsifiers. An industrial mixture is understood to be for example, a commercial product such as Cutina® GMS which is a mixture of glyceryl monostearate and glyceryl distearate.

Additional water-in-oil emulsifiers that can be used especially advantageously include stearyl alcohol, cetyl alcohol, glyceryl monostearate, in particular in the form of the commercial products Cutina® GMS and Cutina® MD (from BASF), glyceryl distearate, glyceryl monocaprinate, glyceryl monocaprylate, glyceryl monolaurate, glyceryl monomyristate, glyceryl monopalmitate, glyceryl monohydroxystearate, glyceryl monooleate, glyceryl monolanolate, glyceryl dimyristate, glyceryl dipalmitate, glyceryl dioleate, propylene glycol monostearate, propylene glycol monolaurate, sorbitan monocaprylate, sorbitan monolaurate, sorbitan monomyristate, sorbitan monopalmitate, sorbitan monostearate, sorbitan sesquistearate, sorbitan distearate, sorbitan dioleate, sorbitan sesquioleate, saccharose distearate, arachidyl alcohol, behenyl alcohol, polyethylene glycol(2)stearyl ether (steareth-2), steareth-5, oleth-2, diglycerol monostearate, diglycerol monoisostearate, diglycerol monooleate, diglycerol dihydroxystearate, diglycerol distearate, diglycerol dioleate, triglycerol distearate, tetraglycerol monostearate, tetraglycerol distearate, tetraglycerol tristearate, decaglycerol pentastearate, decaglycerol pentahydroxystearate, decaglycerol pentaisostearate, decaglycerol pentaoleate, soy sterol, PEG-1 soy sterol, PEG-5 soy sterol, PEG-2 monolaurate and PEG-2 monostearate.

Especially preferred antiperspirant compositions according to the invention are characterized in that at least one water-in-oil emulsifier is included in a total amount of 0.01-10% by weight, especially 0.1-4% by weight and extremely preferably 0.5-3% by weight, based on the total weight of the propellant-free agent.

The HLB values can also be calculated according to Griffin as presented and/or tabulated for example, in RÖMPP Chemie Lexikon [Chemical Lexicon], in particular in the online version of November 2003 and the handbooks by Fiedler, Kirk-Othmer and Janistyn (H. Janistyn, Handbuch der Kosmetika and Riechstoffe [Handbook of Cosmetics and Perfumes], Hüthig-Verlag Heidelberg, 3^{rd} edition, 1978, vol. 1, page 470 and vol. 3, pages 68-78) cited in the online version of RÖMPP under the heading “HLB system”). If different values are given in the literature for the HLB value of a substance, the HLB value that should be used for the teaching according to the invention is the value that comes closest to the value calculated according to Griffin. If an unambiguous HLB value cannot be determined in this way, then the HLB value given by the manufacturer of the emulsifier is to be used for the teaching according to the invention. If this is also impossible, the HLB value shall be determined experimentally.

Additional preferred compositions according to the invention are characterized in that the total amount of nonionic and ionic emulsifiers and/or surfactants with an HLB value of more than 8 amounts to max. 20% by weight, preferably max. 15% by weight, especially preferably max. 10% by weight, especially preferably max. 7% by weight, additionally especially preferably max. 4% by weight and extremely preferably max. 3% by weight, each based on the total agent according to the invention, without taking into account a propellants that might be present.

Especially preferred compositions according to the invention, which are fabricated as a water-in-oil emulsion, preferably also include at least one water-in-oil emulsifier. The at least one water-in-oil emulsifier is preferably present in an amount of 0.1-8% by weight, especially preferably 1.0-2.5% by weight, each based on the total weight of the agent, without taking into account a propellant that might be present.

An especially preferred group of water-in-oil emulsifiers according to the invention are the poly (C₂-C₃)alkylene glycol-modified silicones whose former INCI designation was dimethicone copolyol and whose current INCI designations are PEG-x (with x=2-20, preferably 3-17, especially preferably 11-12), bis-PEG-y dimethicone (with y=3-25, preferably 4-20), PEG/PPG a/b dimethicone (where a and b independently of one another stand for numbers from 2 to 30, preferably 3 to 30 and especially preferably 12 to 20, in particular 14 to 18), bis-PEG/PPG-c/d dimethicone (where c and d independently of one another stand for numbers from 10 to 25, preferably 14 to 20 and especially preferably 14 to 16) and bis-PEG/PPG e/f PEG/PPG g/h dimethicone (where e, f, g, and h independently of one another stand for numbers from 10 to 20, preferably 14 to 18 and especially preferably 16). Especially preferred are PG/PPG-18/18 dimethicone which is available in a 1:9 mixture with cyclomethicone as DC 3225 C and/or DC 5225 C and/or as 25:75 mixture with dimethicone as DC 5227, PG/PPG-4/12 dimethicone which is available under the designation Abil B 8852 as well as bis-PEG/PPG-14/14 dimethicone which is available commercially in a mixture with cyclomethicone as Abil EM 97 (Goldschmidt), bis-PEG/PPG-20/20 dimethicone which is available under the designation Abil B 8832, PEG/PPG-5/3 trisiloxane (Silsoft 305) as well as PEG/PPG-20/23 dimethicone (Silsoft 430 and 440).

Additional W/O emulsifiers preferred according to the invention are poly (C₂-C₃)alkylene glycol-modified silicones which are modified to be hydrophobic with C₄-C₁₈ alkyl groups, especially preferably cetyl PEG/PPG-10/1 dimethicone (previously: cetyl dimethicone copolyol available as Abil EM 90 or in a mixture of polyglycerol 4-isostearate, cetyl PEG/PPG-10/1 dimethicone and hexyllaurate under the brand name Abil WE 09) as well as alkyl methicone copolyols.

Especially preferred compositions according to the invention additionally preferably include at least one skin cooling active ingredient. Suitable skin cooling active ingredients according to the invention include for example, menthol, isopulegol and menthol derivatives for example, menthyl lactate, menthyl glycolate, menthyl ethyl oxamate, menthyl pyrrolidone carboxylic acid, menthyl methyl ether, menthoxypropanediol, menthon glycerol acetal (9-methyl-6-(1-methylethyl)-1,4-dioxaspiro(4.5)decan-2-methanol), monomenthyl succinate and 2-hydroxymethyl-3,5,5-trimethylcyclohexanol. Menthol, isopulegol, menthyl acetate, menthoxypropanediol and menthyl pyrrolidone carboxylic acid as well as mixtures of these substances, in particular mixtures of menthol and menthyl lactate, menthol, menthol glycolate and menthyl lactate, menthol and menthoxypropanediol or menthol and isopulegol are preferred as skin cooling active ingredients.

It is especially preferred according to the invention for at least on skin cooling active ingredient to be present in a total amount of 0.01-2% by weight, especially preferably 0.02-0.5% by weight and extremely preferably 0.05-0.2% by weight, each based on the total weight of the agent, without taking into account a propellant that might be present.

Especially preferred compositions according to the invention which are fabricated as propellant gas-driven aerosol include at least one propellant. Preferred propellants (propellant gases) include propane, propene, n-butane, isobutane, isobutene, n-pentane, pentene, isopentane, isopentene, methane, ethane, dimethyl ether, nitrogen, air, oxygen, nitrous oxide, 1,1,1,3-tetrafluoroethane, heptafluoro-n-propane, perfluoroethane, monochlorodifluoromethane, 1,1-difluoroethane and may be used either individually or in combination. Hydrophilic propellant gases such as carbon dioxide may be used advantageously in the sense of the present invention if the amount of hydrophilic gases is selected to be low and if lipophilic propellant gas (e.g., propane/butane) is present in excess. Especially preferred are propane, n-butane, isobutane and mixtures of these propellant gases. It has been found that the use of n-butane as the only propellant gas according to the invention can be especially preferred.

The amount of propellant is preferably 20-95% by weight, especially preferably 30-85% by weight and extremely preferably 40-75% by weight, each based on the total weight of the preparation consisting of the composition according to the invention (=agent according to the invention) and the propellant.

The pressurized gas container may be containers made of metal (aluminum, tin plate, tin), protected and/or shatterproof plastic or glass coated with plastic on the outside. In choosing the pressurized gas container, compressive strength and fracture strength, corrosion resistance, easy fillability as well as aesthetic aspects, ease of handling, printability, etc. all play a role. Special interprotective coatings ensure the corrosion resistance with respect to the composition according to the invention.

Preferred compositions according to the invention additionally contain at least one water-soluble polyvalent C₂-C₉ alkanol with 2 to 6 hydroxyl groups and/or at least one water-soluble polyethylene glycol with 3 to 20 ethylene oxide units as well as mixtures thereof. These components are preferably selected from 1,2-propylene glycol, 2-methyl-1,3-propanediol, glycerol, 1,2-butylene glycol, 1,3-butylene glycol, 1,4-butylene glycol, pentylene glycols such as 1,2-pentanediol and 1,5-pentanediol, hexanediols such as 1,2-hexanediol and 1,6-hexanediol, hexanetriols such as 1,2,6-hexanetriol, 1,2-octanediol, 1,8-octanediol, dipropylene glycol, tripropylene glycol, diglycerol, triglycerol, erythritol, sorbitol, cis-1,4-dimethylolcyclohexane, trans-1,4-dimethylolcyclohexane, any isomer mixtures of cis- and trans-1,4-dimethylolcyclohexane as well as mixtures of the aforementioned substances. Suitable water-soluble polyethylene glycols are selected from PEG-3, PEG-4, PEG-6, PEG-7, PEG-8, PEG-9, PEG-10, PEG-12, PEG-14, PEG-16, PEG-18 and PEG-20 as well as mixtures thereof, wherein PEG-3 to PEG-8 are preferred.

Preferred antiperspirant agents according to the invention include at least one water-soluble polyvalent C₂-C₉ alkanol with 2-6 hydroxyl groups and/or at least one water-soluble polyethylene glycol with 3-20 ethylene oxide units selected from 1,2-propylene glycol, 2-methyl-1,3-propanediol, glycerol, 1,2-butylene glycol, 1,3-butylene glycol, 1,4-butylene glycol, pentylene glycols, such as 1,2-pentanediol and 1,5-pentanediol, hexanediols such as 1,2-hexanediol and 1,6-hexanediol, hexanetriols such as 1,2,6-hexanetriol, 1,2-octanediol, 1,8-octanediol, dipropylene glycol, tripropylene glycol, diglycerol, triglycerol, erythritol, sorbitol, cis-1,4-dimethylolcyclohexane, trans-1,4-dimethylolcyclohexane, any isomer mixtures of cis- and trans-1,4-dimethylolcyclohexane as well as mixtures of the aforementioned substances.

Especially preferred antiperspirant agents according to the invention include at least one water-soluble polyvalent C₂-C₉ alkanol with 2-6 hydroxyl groups and/or at least one water-soluble polyethylene glycol with 3-20 ethylene oxide units in a total amount of 0.1-30% by weight, preferably 1-20% by weight, especially preferably 5-15% by weight, each based on the total weight of the agent, without taking into account a propellant that might be present.

Especially preferred antiperspirants according to the invention additionally include at least one fragrance. The definition of a fragrance in the sense of the present patent application corresponds to the definition, which is conventionally used by those skilled in the art and can be derived from RÖMPP Chemie Lexikon [Chemistry Lexicon], December 2007 edition. According to this source, a fragrance is a chemical compound with an odor and/or taste, which stimulates the receptors of the hair cells of the olfactory system (adequate stimulus). The required physical and chemical properties include a low molecular weight of max. 300 g/mol, a high vapor pressure, a minimal water solubility and a high lipid solubility as well as a weak polarity and the presence of at least one osmophoric group in the molecule. To differentiate volatile low-molecular substances that are usually not considered to be a fragrance, including those in the sense of the present patent application, but instead are regarded and used primarily as solvents such as ethanol, propanol, isopropanol and acetone, from the fragrances according to the invention, the fragrances according to the invention have a molecular weight of 74 to 300 g/mol, include at least one osmophoric group in the molecule and have an odor and/or taste, i.e., they stimulate the receptors of the hair cells of the olfactory system.

Perfumes, perfume oils or perfume oil ingredients may be used as fragrances. According to the invention, perfume oils and/or scents may include individual fragrance compounds, for example, the synthetic products of the type of esters, ethers, aldehydes, ketones, alcohols and hydrocarbons. Fragrance compounds of the ester type include, for example, benzyl acetate, phenoxyethyl isobutyrate, p-tert-butylcyclohexyl acetate, linalyl acetate, dimethylbenzylcarbinyl acetate (DMBCA), phenylethyl acetate, benzyl acetate, ethylmethylphenyl glycinate, allylcyclohexyl propionate, styrallyl propionate, benzyl salicylate, cyclohexyl salicylate, floramate, melusate and jasmecyclate. The ethers include, for example, benzyl ethyl ether and ambroxan. The aldehydes include, for example, the linear alkanols with 8 to 18 carbon atoms, citral, citronellal, citronellyl oxyacetaldehyde, cyclamen aldehyde, lilial and bourgeonal. The ketones include, for example, the ionones, α-isomethyl ionone and methyl cedryl ketone. The alcohols include, for example, anethol, citronellol, eugenol, geraniol, linalool, phenylethyl alcohol and terpineol. The hydrocarbons include mainly the terpenes, such as limonene and pinene. However, mixtures of different fragrances which jointly create an appealing scent note are preferred.

Especially preferred antiperspirant agents according to the invention include at least one fragrance component in a total amount of 0.0001 to 10% by weight, preferably 0.5-7% by weight, extremely preferably 1-6% by weight, each based on the total weight of the agent, without taking into account a propellant that might be present.

In a preferred embodiment according to the invention, the at least one antiperspirant aluminum salt is suspended undissolved in at least one oil that is liquid under standard conditions. For better applicability, at least one lipophilic thickening agent is added as a suspension aid to this suspension. Additional preferred compositions according to the invention are therefore characterized in that they include at least one lipophilic thickener. Preferred lipophilic thickeners according to the invention are selected from hydrophobized clay minerals and pyrogenic silicic acids. Of these hydrophobized clay minerals are especially preferred.

Preferred hydrophobized clay minerals are selected from hydrophobized montmorillonite, hydrophobized hectorites and hydrophobized bentonites, especially preferably from disteardimonium hectorites, stearalkonium hectorites, quaternium-18 hectorites and quaternium-18 bentonites. The commercial thickeners supply these hydrophobized clay minerals in the form of a gel in an oil component preferably in cyclomethicone and/or a non-silicone oil component such as propylene carbonate. The gel is formed by adding small amounts of activators such as ethanol or propylene carbonate in particular but also water. Such gels are available under the brand names Bentone® or Thixo-Gel, for example. Preferred compositions according to the invention include at least one activator in a total amount of 0.1-3% by weight, preferably 0.3-1.6% by weight, each based on the total weight of the propellant-free composition according to the invention. Additional preferred compositions according to the invention include at least one activator selected from ethanol, propylene carbonate and water as well as mixtures thereof in a total amount of 0.1-3% by weight, preferably 0.3-1.6% by weight each based on the total weight of the propellant-free composition according to the invention. Preferred compositions according to the invention are characterized in that they include at least one hydrophobized clay mineral in a total amount of 0.5-10% by weight, preferably 1-7% by weight, especially preferably 2-6% by weight, extremely preferably 3-5% by weight, each based on the total weight of the propellant-free composition according to the invention.

Additional lipophilic thickeners that are preferred according to the invention are selected from pyrogenic silicic acids, for example, the commercial products of the Aerosil® series from Evonik Degussa. Especially preferred are the hydrophobized pyrogenic silicic acids, especially preferably silica silylate and silica dimethyl silylate.

Preferred compositions according to the invention are characterized in that they include at least one pyrogenic silicic acid, preferably at least one hydrophobized pyrogenic silicic acid in a total amount of 0.5-10% by weight, preferably 0.8-5% by weight, especially preferably 1-4% by weight, extremely preferably 1.5-2% by weight, each based on the total weight of the propellant-free composition according to the invention.

Additional preferred compositions according to the invention are characterized in that they include at least one hydrophobized pyrogenic silicic acid and at least one hydrophilic silicic acid.

Another subject matter of the present patent application is a method for nontherapeutic cosmetic antiperspirant treatment of the body, in which an antiperspirant cosmetic agent is applied to the skin, in particular to the axillary skin, this antiperspirant cosmetic agent comprising, in a cosmetically tolerable vehicle, at least one antiperspirant zirconium-free aluminum salt in a total amount of 2-40% by weight, preferably 8-35% by weight, especially preferably 10-28% by weight and extremely preferably 12-20% by weight, wherein the amounts in percent by weight are based on the total weight of the active substance (USP) free of ligands and free of water of crystallization in the composition, plus at least one hydroxycarboxylic acid of the formula (HCS-1):

HOOC—(CHOH)ₙ—CHOH—R (HCS-1)

where n is an integer in the range of 1 to 10, and the substituent R is selected from —H and —COOH, wherein the hydroxycarboxylic acid may be present in free form, in salt form or as a lactone and wherein the total amount of hydroxycarboxylic acid(s) of the formula (HCS-1), optionally in salt form and/or lactone form, is 0.3-5% by weight, preferably 0.5-3% by weight, especially preferably 1-2.5% by weight, wherein the amounts in percent by weight are each based on the total weight of the agent, without taking into account a propellant that might be present.

With respect to preferably embodiments of the method according to the invention, what was said about the agents and products according to the invention is also applicable mutatis mutandis.

Another subject matter of the present patent application is a method for preventing and/or reducing textile discoloration and/or textile spots, wherein the method comprises the following method steps:
(a) producing an antiperspirant cosmetic agent by mixing at least one antiperspirant zirconium-free aluminum salt in a total amount of 2-40% by weight, preferably 8-35% by weight, especially preferably 10-28% by weight and extremely, preferably 12-20% by weight, wherein the amounts by weight are based on the total weight of the active substance (USP) free of ligands and free of water of crystallization in the agent, with a cosmetically tolerable vehicle and with at least one hydroxycarboxylic acid of the formula (HCS-1):

HOOC—(CHOH)ₙ—CHOH—R (HCS-1)
where n is an integer in the range of 1 to 10, and the substituent R is selected from —H and —COOH, wherein the hydroxycarboxylic acid may be present in free form, in salt form or as a lactone and wherein the total amount of hydroxycarboxylic acid(s) of the formula (HCS-1), optionally in salt form and/or lactone form, is 0.3-5% by weight, preferably 0.5-3% by weight, especially preferably 1-2.5% by weight, wherein the amounts in percent by weight are each based on the total weight of the propellant-free agent,
(b) filling the agent into a package, selected from a pump spray container, a squeeze container and a spray can including at least one propellant,
(c) applying the antiperspirant cosmetic agent to the skin in particular to the axillary skin,
(d) wearing an item of textile clothing over the treated skin and
(e) washing the item of textile clothing, in particular washing the item of textile clothing several times, wherein after washing, in particular after washing several times, reduced textile discoloration and/or textile spots or none at all appear.

With respect to preferred embodiments of the method according to the invention for preventing and/or reducing textile discolorations and/or textile spots, what was said about the agents and products according to the invention is also applicable mutatis mutandis.

### Experimental Part

### Waterless Antiperspirant Compositions

A suspension consisting of 14.3% by weight activated aluminum chlorohydrate, 67.9% by weight 2-ethylhexyl palmitate, 5.4% by weight triethyl citrate, 3.9% by weight Bentone 38 V CG, 1.3% by weight propylene carbonate and 7.2% by weight perfume was prepared as a reference product not according to the invention (V−1).

TABLE 1
Test products and reference products used
(quantitative amounts in percent by weight)

| | V-1 | E-1 | E-2 | E-3 |
|---|---|---|---|---|
| Activated aluminum chlorohydrate | 14.3 | 14.3 | 14.3 | 14.3 |
| (AACH) | | | | |
| 2-Ethylhexyl palmitate | 67.9 | 65.9 | 65.9 | 65.9 |
| Triethyl citrate | 5.4 | 5.4 | 5.4 | 5.4 |
| Bentone 38 V CG | 3.9 | 3.9 | 3.9 | 3.9 |
| Propylene carbonate | 1.3 | 1.3 | 1.3 | 1.3 |
| Perfume | 7.2 | 7.2 | 7.2 | 7.2 |
| D-Gluconic acid 5-lactone (finely | — | 2 | — | — |
| granulated) | | | | |
| Sodium gluconate (finely granulated) | — | — | 2 | — |
| D-Tetrahydroxyadipic acid mixture, | — | — | — | 2 |
| potassium salts | | | | |

### Experimental Procedure

0.3 gram of the respective test product or reference product was applied directly to a 10×10 cm² piece of light blue cotton cloth (polo jersey, woven) which was attached to a waffle piqué hand towel. After a waiting time of 1 hour, 1 mL of an artificial perspiration mixture (MgCl₂, CaC₁₂, KCl, NaCl, Na₂SO₄, NaH₂PO₄, glycine, glucose, lactic acid, urea, pH 5.2) and the textile was washed after a waiting time of 24 hours (aging) in a standardized household laundry process (Miele W 1714) and machine dried (Miele T 7644C).

TABLE 2
Additional conditions of the washing experiments

| | | |
|---|---|---|
| Washing machine load | 3.5 | kg |
| Amount of water | 17 | L |
| Temperature | 40° | C. |
| Time of main wash cycle | 1 | h |

| | |
|---|---|
| Prewash | none |
| Final-rinse | 4× |
| Detergent | Spee color gel |
| | Lot: HH06.1.1UWM1.08:58 |
| Weighed amount of detergent | 75 mL (70 g) |
| Fabric softener | none |
| Dryer program | extra dry cotton |

The application of the product and the washing were repeated a total of eight times with the same textile. The soiling of the textile was evaluated visually by trained laboratory personnel on the basis of reference examples. The scale ranged from 0 (no spots) to 4 (very heavy formation of spots). The evaluation was performed immediately after conclusion of the laundry series.

TABLE 3
Results of the visual evaluation of residue

| | after 8 washings | | |
|---|---|---|---|
| Product | White | greasy | yellow |
| V-1 | 0 | 4 | 0 |
| E-1 | 0 | 2 | 0 |
| E-2 | 0 | 2.5 | 0 |
| E-3 | 0 | 3 | 0 |

The test products according to the invention, each with 2% by weight of a hydroxycarboxylic acid of the formula (HCS-1) used according to the invention, showed a definitely reduced formation of greasy spots on light blue textile (Table 3) in comparison with the reference formulation V-1 without HSC-1 (Table 1).

### Antiperspirant Compositions Including Water

Aqueous solutions consisting of 20% by weight aluminum chlorohydrate, 2% by weight of the hydroxycarboxylic acid of the formula (HCS-1) used according to the invention and to be tested as well as 78% by weight water were prepared (E-4, E-5, E-6). Such solutions are representative of antiperspirant emulsions including water, among others.

A solution consisting of 20% by weight aluminum chlorohydrate and 80% water was used as the reference product not according to the invention (V).

TABLE 4
Test products and reference products used
(quantitative amounts in percent by weight)

| | V-2 | E-4 | E-5 |
|---|---|---|---|
| Aluminum chlorohydrate (ACH) | 20 | 20 | 20 |
| Water | 80 | 78 | 78 |
| D-Gluconic acid 5-lactone(finely granulated) | — | 2 | — |
| D-Tetrahydroxyadipic acid mixture, potassium | — | — | 2 |
| salts | | | |

The laundry experiments were conducted like the test series with the waterless compositions.

TABLE 5
Results of the visual evaluation of residue

| | after 8 washings | | |
|---|---|---|---|
| Product | white | greasy | yellow |
| V-2 | 4 | 0 | 0 |
| E-4 | 3 | 0 | 0 |
| E-5 | 3 | 0 | 0 |

The test products E-4 and E-5 according to the invention, each with 2% by weight of a hydroxycarboxylic acid of the formula (HCS-1) used according to the invention showed a definitely reduced formation of white spots on light blue textile (Table 5) in comparison with the reference formulation V-2 without HSC-1 (Table 1).

### Exemplary Embodiments

The following examples of formulations should illustrate the subject matter of the invention, without limiting it to these.

TABLE 6
Translucent antiperspirant microemulsions sprayable
as pump sprays (amounts in percent by weight)

| | 1.1 | 1.2 | 1.3 | 1.4 | 1.5 | 1.6 |
|---|---|---|---|---|---|---|
| Plantaren ® 1200 | 1.71 | 1.71 | — | 1.71 | 1.71 | — |
| Plantaren ® 2000 | 1.14 | 1.39 | 2.40 | 1.14 | 1.39 | 2.40 |
| Glycerol | 0.71 | 0.71 | — | 0.71 | 0.71 | — |
| monooleate | | | | | | |
| Dioctyl ether | 4.00 | 4.00 | 0.09 | 4.00 | 4.00 | 0.09 |
| Octyl dodecanol | 1.00 | 1.00 | 0.02 | 1.00 | 1.00 | 0.02 |
| Perfume oil | 1.00 | 1.00 | 1.00 | 1.00 | 1.00 | 1.00 |
| Aluminum | 8.00 | 5.00 | 5.00 | 15.00 | 10.00 | 12.00 |
| chlorohydrate | | | | | | |
| 1,2-Propylene | 5.00 | 5.00 | — | 5.00 | 5.00 | 5.00 |
| glycol | | | | | | |
| Glycerol | — | — | 5.00 | — | — | — |
| 2-Benzylheptan- | 0.5 | — | — | 0.5 | 0.5 | — |
| 1-ol | | | | | | |
| Triethyl citrate | — | 0.5 | 0.5 | 0.2 | — | — |
| Triclosan | — | — | — | — | — | 0.5 |
| D-Gluconic | 1.0 | 2.0 | 2.0 | 2.5 | 0.5 | 3.0 |
| acid 5-lactone | | | | | | |
| Water | to 100 | to 100 | to 100 | to 100 | to 100 | to 100 |

TABLE 7
Water-in-oil emulsions according to the invention (all quantitative
amounts in percent by weight, based on the total weight of
the water-in-oil emulsion without propellant)

| | 2.1 | 2.2 | 2.3 | 2.4 | 2.5 |
|---|---|---|---|---|---|
| Aluminum chlorohydrate | 33 | 33 | 33 | 33 | 33 |
| C₁₀-C₁₃-Isoalkane | 8.9 | 8.9 | 8.9 | 8.9 | 8.9 |
| PEG/PPG-18/18 | 1.4 | 1.4 | 1.4 | 1.4 | 1.4 |
| dimethicone* | | | | | |
| Isoceteth-20 | 0.50 | 0.50 | 0.50 | 0.50 | 0.50 |
| Dimethicone* | 4.2 | 4.2 | 4.2 | 4.2 | 4.2 |
| Isopropyl myristate | 9.0 | 9.0 | 9. | 9.0 | 9.0 |
| 1,2-Propanediol | 7.0 | 25 | 25 | 25 | 25 |
| Phenoxyethanol | 0.50 | 0.50 | 0.50 | 0.50 | 0.50 |
| Perfume | 2.5 | 2.5 | 2.5 | 2.5 | 2.5 |
| D-Gluconic acid | 2.0 | 0.5 | 3.0 | — | — |
| 5-lactone | | | | | |
| Sodium gluconate | | | | 3 | |
| Glucaric acid | | | | | 1.5 |
| L-Menthol | 0.4 | 0.3 | — | — | |
| trans-Anethol | — | 0.3 | — | — | |
| Eucalyptus | — | 0.3 | — | — | |
| Water | to 100 | to 100 | to 100 | to 100 | to 1000 |

*from Dow Corning ES-5227 DM

The antiperspirant water-in-oil emulsions 2.1, 2.2 to 2.5 according to the invention were used to fill an aluminum an coated on the inside with epoxy phenolic coating in a weight ratio of propellant (butane/propane/isobutane mixture) to emulsion of 80:20 and/or 85:15 and/or 60:40 and/or 10:90.

The exemplary compositions according to the invention were sprayed on the axillary skin.

TABLE 8
Antiperspirants sprays in the form of a water-in-oil emulsion (amounts
in percent by weight based on propellant-including composition)

| | 3.1 | 3.2 |
|---|---|---|
| Aluminum chlorohydrate 50% in water (Locron L) | 10.0 | 10.0 |
| Pionier 2094 | 1.7 | 1.7 |
| Dow Corning ES-5227 DM formulation aid | 1.0 | 1.0 |
| 1,2-Propylene glycol | 5.0 | 5.0 |
| D-Gluconic acid 5-lactone | 2.0 | 0.5 |
| EDTA | — | 0.01 |
| Propane | 12.0 | 12.0 |
| Butane | 68.0 | 68.0 |
| Perfume | 1.0 | 1.0 |
| Isopropyl myristate | to 100 | to 100 |

TABLE 9
Suspensions for spraying as antiperspirant spray

| | 4.1 | 4.2 | 4.3 | 4.4 |
|---|---|---|---|---|
| Perfume | 5.00 | 5.00 | 5.00 | 5.00 |
| Aluminum chlorohydrate | 30.00 | 35.00 | 35.00 | 35.00 |
| (activated) | | | | |
| D-gluconic acid 5-lactone | 2.00 | — | — | — |
| D-tetrahydroxyadipic acid | — | 2.00 | — | — |
| mixture, potassium salts | | | | |
| Sodium gluconate | — | — | 2.0 | — |
| D-potassium galactarate | — | — | — | 2.0 |
| Isopropyl palmitate | 5.00 | 5.00 | — | — |
| C₁₂₋₁₅ alkyl benzoate | — | — | 5.00 | 5.00 |
| Disteardimonium hectorite | 4.50 | 3.90 | 4.00 | 3.80 |
| Propylene carbonate | 1.50 | 1.30 | 1.30 | 1.30 |
| Cyclopentasiloxane | to 100 | to 100 | to 100 | to 100 |

The compositions 4.1 to 4.4 according to the invention were used to fill spray cans made of aluminum, coated on the inside and pressurized with an isobutane/butane/propane propellant mixture in a suspension/propellant weight ratio of 25:75, 22:78, 20:80 and 18:82.

List of raw materials used

| | | Supplier/ |
|---|---|---|
| Component | INCI | Manufacturer |
| Dow Corning ES-5227 | Dimethicone, PEG/PPG- | Dow Corning |
| DM formulation aid | 18/18 dimethicone in | |
| | a weight ratio 3:1 | |
| Locron L (ACH solution | Aluminum chlorohydrate | Clariant |
| 50%) | | |
| Plantaren ® 1200 | Lauryl glucoside, approx. | BASF |
| | 50% AS | |
| Plantaren ® 2000 | Decyl glucoside, approx. | BASF |
| | 50% AS | |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic product, comprising:
i. a package, selected from a pump spray container, a squeeze container, a spray can including at least one propellant and
ii. an antiperspirant cosmetic agent contained therein for spray application, including in a cosmetically tolerable vehicle:
a. at least one antiperspirant zirconium-free aluminum salt in a total amount of 2-40% by weight, wherein the percent by weight are based on the total weight of the active substance (USP), which is free of ligands and free of water of crystallization in the agent,
and in addition to that
a) at least one hydroxycarboxylic acid of the formula (HCS-1):
HOOC—(CHOH)ₙ—CHOH—R (HCS-1)
wherein n denotes an integer in the range of 1 to 10, and the substituent R is selected from —H and —COOH, wherein the hydroxycarboxylic acid may be present in free form, in salt form or as a lactone and wherein the total amount of hydroxycarboxylic acid(s) of the formula (HCS-1),
wherein the amounts given in percent by weight are each based on the total weight of the agent, without taking into account the propellant that is optionally present.

2. The cosmetic product according to claim 1, wherein the at least one antiperspirant zirconium-free aluminum salt comprises 8 wt % to 35 wt % of the antiperspirant cosmetic agent.

3. The cosmetic product according to claim 1, wherein the hydroxycarboxylic acid(s) of the formula (HCS-1) is a salt form and/or lactone form.

4. The cosmetic product according to claim 1, wherein the hydroxdycarboxlic acid(s) of the formula (HCS-1) comprises 0.3-5% by weight of the total weight of the antiperspirant cosmetic agent.

5. The cosmetic product according to claim 1, wherein the at least one hydroxycarboxylic acid of formula (HCS-1) is not an ingredient of the antiperspirant aluminum salt.

6. The cosmetic product according to claim 1, wherein the at least one hydroxycarboxylic acid of the formula (HCS-1) is selected from the group consisting of hydroxymalonic acid, dihydroxysuccinic acid, trihydroxyglutaric acid, tetrahydroxyadipic acid, gluconic acid, glucoheptonic acid, mannonic acid and ribaric acid, all enantiomers of the aforementioned acids as well as the physiologically tolerable salts and the lactones of these acids, and combinations thereof.

7. The cosmetic product according to claim 1, wherein the at least one hydroxycarboxylic acid of formula (HCS-1) is D-gluconic acid 5-lactone.

8. The cosmetic product according to claim 1, wherein the at least one hydroxycarboxylic acid of the formula (HCS-1) is selected from the group consisting of glucaric acid (R=—COOH and n=4), gularic acid (R=—COOH and n=4) and galactaric acid (mucic acid, R=—COOH and n=4), the salts of these acids, and mixtures thereof.

9. The cosmetic product according to claim 1, wherein the at least one hydroxycarboxylic acid of the formula (HCS-1) is selected from the group consisting of sodium salts and/or potassium salts of glucaric acid, gularic acid and/or galactaric acid as well as D-gluconic acid 5-lactone and mixtures of the aforementioned compounds.

10. The cosmetic product according to claim 1, wherein the physiologically tolerable salts are selected from the sodium salts and the potassium salts of the at least one hydroxycarboxylic acid of the formula (HCS-1).

11. The cosmetic product according to claim 1, wherein the hydroxycarboxylic acid of the formula (HCS-1) is D-gluconic acid 5-lactone and comprises 0.3-5% by weight, of the antiperspirant cosmetic agent without the optionally present propellant.

12. The cosmetic product according to claim 1, wherein the antiperspirant cosmetic agent further comprises at least one cosmetic oil which is not a perfume and is not an essential oil.

13. The cosmetic product according to claim 1, wherein the antiperspirant cosmetic agent includes up to a maximum of 10% by weight free water based on the total weight of the antiperspirant cosmetic agent, without the optional propellant.

14. The cosmetic product according to claim 1, wherein the antiperspirant cosmetic agent includes 15-96 wt % by weight free water, based on the total weight of the antiperspirant cosmetic agent without the optional propellant.

15. The cosmetic product according to claim 1, wherein the antiperspirant cosmetic agent further comprises at least one emulsifier and/or at least one surfactant.

16. A method for preventing or reducing textile discolorations and/or textile spots, wherein the method comprises the following method steps:
a) producing an antiperspirant cosmetic agent by mixing at least one antiperspirant zirconium-free aluminum salt in a total amount of 2-40% by weight, wherein the amounts in percent by weight are based on the total weight of the active substance (USP), which is free of ligands and free of water of crystallization, in the agent and is present in the propellant-free agent, with a cosmetically tolerable vehicle and with at least one hydroxycarboxylic acid of the formula (HCS-1):
HOOC—(CHOH)ₙ—CHOH—R (HCS-1)
where n denotes an integer in the range of 1 to 10, and the substituent R is selected from —H and —COOH, wherein the hydroxycarboxylic acid may be present in free form, in salt form or as a lactone and wherein the hydroxycarboxylic acid is present in a total amount of 0.3-5% by weight, wherein the amounts by weight are each based on the total weight of the agent,
b) bottling the agent in a package, selected from a pump spray container, a squeeze container and a spray can containing at least one propellant,
c) applying the antiperspirant cosmetic agent to a skin surface,
d) wearing an item of textile clothing over the treated skin, and
e) washing the item of textile clothing, wherein after washing, reduced textile discolorations and/or textile spots or none at all appear.
